(19) United States
(12) Patent Application Publication
(10) Pub. No.: US 2018/0245073 A1
(43) Pub. Date: Aug. 30, 2018

(54) REGULATABLE EXPRESSION USING ADENO-ASSOCIATED VIRUS (AAV)

(71) Applicant: VOYAGER THERAPEUTICS, INC., CAMBRIDGE, MA (US)

(72) Inventors: Robert Kotin, Boston, MA (US); James McLaughlin, Cambridge, MA (US); Jinzhao Hou, Belmont, MA (US)

(21) Appl. No.: 15/553,021

(22) PCT Filed: Feb. 23, 2016
(86) PCT No.: PCT/US2016/019066

§ 371 Date: Aug. 23, 2017

Related U.S. Application Data
(60) Provisional application No. 62/119,416, filed on Feb. 23, 2015.
(60) Provisional application No. 62/199,399, filed on Jul. 31, 2015.

(51) Int. Cl. C12N 15/11; C12N 9/22

(52) U.S. Cl. CPC C12N 15/11; C12N 2310/20; C12N 2750/14142; C12N 9/22

(57) ABSTRACT

The present invention relates to CRISPR-AAV vectors and viral particles which exhibit self-regulatory or regulatable features.

### CROSS REFERENCE TO RELATED APPLICATIONS

This application claims the benefit of U.S. Provisional Patent Application No. 62/119,416, entitled Regulatable Expression Using Adeno-Associated Virus (AAV), filed Feb. 23, 2015, and U.S. Provisional Patent Application No. 62/199,399, entitled Regulatable Expression Using Adeno-Associated Virus (AAV), filed Jul. 31, 2015, the contents of each of which are herein incorporated by reference in their entirety.

### REFERENCE TO THE SEQUENCE LISTING

The present application is being filed along with a Sequence Listing in electronic format. The Sequence Listing file, entitled 1501PCTSEQLST.txt, was created on Feb. 22, 2016 and is 2,281,497 bytes in size. The information in electronic format of the Sequence Listing is incorporated herein by reference in its entirety.

### FIELD OF THE INVENTION

The present invention relates to compositions, methods and processes for the design, preparation, manufacture and/or formulation of recombinant parvovirus, e.g. adeno-associated virus (AAV), particles having one or more CRISPR regulatable expression elements and methods of using the same.

### BACKGROUND OF THE INVENTION

First identified in 1987, the CRISPR (Clustered Regularly Interspersed Short Palindromic Repeats) system functions as an adaptive immune response defense in the genomes of several bacteria and Archaea.

In bacteria, CRISPR, along with CRISPR-associated or cas genes, function in association with non-coding RNAs to recognize and destroy foreign DNA and to ensure survival against subsequent invasions by a similar pathogen, whether a virus or plasmid. Three types of CRISPR systems have been identified in bacteria with the Type II system being the most widely explored.

The Type II natural RNA-guided DNA nuclease system includes the Cas9 nuclease (also known as Csn1 and formerly known as Cas5) and two small RNAs known as “crRNA” or CRISPR RNA and “tracrRNA” or trans-activating CRISPR RNA. Both are processed from the clustered repeats encoded in the bacterial host genome. The type II CRISPR system requires both the crRNA and the tracrRNA to be functional. In this system, the crRNA associates with the cas9 endonuclease and acts as a hybridization strand providing localization to the complementary target DNA while the tracrRNA associates with the crRNA through partial hybridization and has been shown to be necessary for Cas9 complex binding to the target dsDNA. Once associated with the target dsDNA site, the cas9 enzyme cleaves both strands of the dsDNA thereby destroying the invading organism.

The Type II system is exemplified by the systems found in Streptococcus pyogens and Streptococcus thermophilus. Here the effector complex involves a single Cas9 protein. Early studies of CRISPR-dependent immunity in Streptococcus thermophilus were performed by Barrangou and colleagues (Barrangou et al., Science; 2007; 315:1709-12; Horvath and Barrangou, Science, 2010; 327:167-170). In the S. thermophilus strain, the tracrRNA of approximately 65 nucleotides co-purifies with the Cas9 protein and a 42 nucleotide crRNA (Karvelis et al., RNA Biology, 2013; 10:5: 841-851). Sapranauskas et al. demonstrated the transfer of the S. thermophilus CRISPR3/Cas system into E. coli and that this transfer could provide protection against plasmid transformation. It was also shown that the protection was sequence specific (NAR, 2011; 39(21): 9275-9282).

Studies of the maturation process of the crRNA and tracrRNA in S. pyogens illustrated the necessity of the tracrRNA for maturation of the functional complex and the involvement of an RNase III (Deltcheva, et al., Nature, 2011; 471:602-607). The work of Deltcheva et al. led to the work of Jinek, et al. (Science, 2012; 337:816-821) showing that the crRNA and tracrRNA could be combined into one chimeric RNA molecule to produce a functional ribonucleoprotein complex which could cleave a plasmid or oligonucleotide duplex bearing the required protospacer and PAM. Jinek et al. showed that each Cas9 domain cleaved only one strand of the dsDNA duplex and that point mutations in conserved catalytic amino acids of the two domains (D10A and H840A) resulted in the determination that the HNH domain (mutation H840A) cleaves the strand complementary to the crRNA (or template strand) while the RuvC-like domain (mutation D10A) cleaves the non-complementary or displaced strand (or non-template strand) which comprises the protospacer.

It has since been discovered that Type II CRISPR nuclease-guided cleavage of dsDNA can be reprogrammed to work in higher organism by providing a Cas9 enzyme and altering the features of the two small RNAs associated with the Cas enzyme. In higher organisms such as in mammalian cells, targeting and cleavage of the endogenous or genomic dsDNA triggers the cell's natural repair mechanisms through either non-homologous end joining (NHEJ) or homology directed repair (HDR) pathways, thereby editing the target genomic site.

This observation inspired a series of studies exploring the requirements of tracrRNA size, DNA binding and hybridization, mutant Cas9 enzymes, delivery of the enzyme and RNA molecules to mammalian cells, localization to the nucleus, off-target effects, the specificity of genome editing and multiplexing target sites (Karvelis et al., RNA Biology, 2013; 10:5; 841-851; Gasiunas et al., PNAS, USA 2012; 109:E2579-E2586; Cong et al., Science, 2013; 339: 819-823; Mali, et al., Science; 2013; 339: 823-826; Hwang et al., Nat. Biotechnol., 2013; doi 10.1038/nbt.2501; Cho et al., Nat. Biotechnol., 2013; doi 10.1038/nbt.2507; Jiang et al., Nat. Biotechnol., 2013; 31; 233-241; Fu et al., Nat. Biotechnol. 2013; doi: 10.1038/nbt2623; and Chylinski et al., RNA Biology; 2013; 10:5; 726-737).

The use of the Cas9 CRISPR system in mice has been studied by Shen and Wang et al., (Cell, 2013; 153:910-918). Shen et al. targeted a GFP transgene in the mouse genome by administering a Cas9 mRNA and pre-annealed crRNA-tracrRNA chimera to mouse embryos. They showed site specific cleavage in a chromosomal locus. Wang et al. explored triggering homologous recombination in the mouse and utilized a Cas9 mRNA and chimeric crRNA-tracrRNA. In these studies, conversion of an EcoRV site to an EcoRI restriction sites was successful upon a two base pair insertion. Still neither group demonstrated insertion of a larger polynucleotide.

Cleavage deficient Cas9 enzymes of Streptococcus pyogens and S. thermophilus have been explored further by Sapranauskas et al., (NAR, 2011; 39(21); 9275-9282), Qi et al., (Cell, 2013; 152:1173-1183) and Bikard et al., (NAR, 2013; 1-9) where the effects on transcription modulation including upregulation and silencing were investigated.

Studies in organisms other than bacteria include those in yeast (DiCarlo et al., NAR; 2013; 41:4336-4343), Drosophila (Gratz; Yu et al., Genetics, 2013; doi10.1534/genetics.113.153825) and Zebrafish (Hwang et al., Nat. Biotechnol. Doi 10.1038/nbt.2501).

On this background there remains, however, much work to be done to define the optimized components of a system which will be effective in exploiting the cas9 CRISPR system as a tool for research, diagnostics and therapeutics with the ultimate goal of target-specific genome editing.

And while other genome editing technologies such as zinc finger (ZFNs), transcription activator-like effector nucleases (TALENs) and homing meganuclease have recently been utilized to edit DNA in hopes of repairing genomic defect, these suffer from lack of scalability, difficulty in individualized engineering and the expense of designing a unique complex for each locus to be altered.

The present invention addresses the need for new technologies for treating genetic disorders caused by abnormalities in the genome whether heritable or acquired, monogenic or multifactorial, by providing AAV-based compositions and complexes which go beyond those of the art.

The present invention provides recombinant adeno-associated virus particles having at least one CRISPR regulatable element. These elements when used in association with AAV technology allow, for the first time, the regulatable tuning of payload expression from a viral genome delivered by an AAV particle and consequently the ability to produce gene editing events at discrete loci while being able to control or limit the duration of the constitutive nature of payload expression from an AAV viral genome.

While exemplified in the AAV system, polynucleotides, vectors, and/or particles of the invention include, but are not limited to vector genomes of any of the AAV serotypes or other parvoviral viral delivery vehicles or lentivirus, etc.

### SUMMARY OF THE INVENTION

The present invention provides CRISPR-AAV vectors and viral particles for the regulatable expression of genes. Such regulation can be the inhibition of gene expression, gene replacement or gene activation. Such outcomes are achieved by utilizing CRISPR regulatable elements encoded in the polynucleotide of the AAV particles in such a manner as to tune or control the level or degree of expression of the payload (whether a polynucleotide useful for gene knockdown or for gene replacement, activation, or inhibition) encoded by the AAV viral genome.

Manufacture of such particles is performed in insect cells, e.g., sf9 cells and utilize a heterologous promoter such that the autocatalytic features of the polynucleotide will not result in self destruction of the molecule as manufactured.

In one embodiment the present invention is a composition comprising an AAV particle comprising a polynucleotide encoding at least one payload and at least one CRISPR regulatable element.

In another embodiment the present invention is a method of synthesizing a CRISPR-AAV particle comprising a) introducing into competent bacterial cells i) a payload construct vector comprising a payload and one or more CRISPR regulatable elements flanked on each side by a parvoviral ITR sequence to produce a payload construct expression vector; and ii) one or more viral construct vector(s) comprising parvoviral rep and/or cap gene sequences under the control of one or more regulatory elements to produce a viral construct expression vector; b) introducing into viral replication cells i) the payload construct expression vector produced in step (a.i) to produce a payload construct particle; and ii) the viral construct expression vector(s) produced in step (a.ii) to produce a viral construct particle; and c) co-infecting a viral replication cell with the payload construct viral particle produced in step (b.i) and the one or more viral construct viral particle(s) of step (b.ii) to produce a CRISPR-AAV particle.

In another embodiment the present invention is a CRISPR-AAV particle comprising an AAV polynucleotide, the AAV polynucleotide comprising: (a) at least one payload, and (b) at least one CRISPR regulatable element

In another embodiment the present invention is a method of treating a CNS disorder in a subject in need thereof, the method comprising administering to the subject a therapeutically effective amount of a CRISPR-AAV particle comprising an AAV polynucleotide, the AAV polynucleotide comprising: (a) at least one payload, and (b) at least one CRISPR regulatable element.

In one embodiment, provided are methods of regulating the expression of a protein of interest using a CRISPR AAV particle comprising an AAV polynucleotide.

In one aspect, the AAV polynucleotide may have at least one payload and at least one CRISPR regulatable element such as, but not limited to, a DNA binding domain which may be coupled with a transactivation domain. The regulatable element may be located in the VP2 capsid and may increase the expression of a protein of interest in a burst like fashion. The increase may be for at least 2 hours or may be for at least 6 hours.

In another aspect, the AAV polynucleotide may have at least one payload and at least one CRISPR regulatable element such as, but not limited to, a cas9 endonuclease fused to a destabilizing domain. The destabilizing domain may be a destabilizing domain from a protein family such as, but not limited to, FK506 Binding Protein (FKBP), E. coli dihyrofolate reductase (DHFR), mouse ornithine decarboxylase (MODC), and estrogen receptors (ER). As a non-limiting example, the destabilizing domain is from an estrogen receptor protein.

### DETAILED DESCRIPTION OF THE INVENTION

The present invention relates to compositions, methods and processes for the design, preparation, manufacture and/or formulation of recombinant adeno-associated virus (AAV) particles having one or more CRISPR regulatable expression elements and methods of using the same.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

Viruses of the Parvoviridae family are small non-enveloped icosahedral capsid viruses characterized by a single stranded DNA genome. Parvoviridae family viruses consist of two subfamilies: Parvovirinae, which infect vertebrates, and Densovirinae, which infect invertebrates. This virus family is used as biological tools due to a relatively simple structure that may be easily manipulated with standard molecular biology techniques. The genome of the virus may be modified to contain a minimum of components for the assembly of a functional recombinant virus, or viral particle, which is loaded with or engineered to express or deliver a desired nucleic acid construct or payload, e.g., a transgene, polypeptide-encoding polynucleotide or modulatory nucleic acid, which may be delivered to a target cell, tissue or organism.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which is incorporated by reference in its entirety.

The Parvoviridae family comprises the Dependovirus genus which includes adeno-associated viruses (AAV) capable of replication in vertebrate hosts including, but not limited to, human, primate, bovine, canine, equine, and ovine species.

Described herein are compositions, methods, processes, kits and devices for the design, preparation, manufacture and/or formulation of AAV particles for altering the genomic state or status of a cell, tissue, organ or organism, herein referred to as “regulatable AAV expression particles.” Such AAV particles comprise one or more CRISPR regulatable expression elements and hence are termed “CRISPR-AAV” particles. As used herein, a “CRISPR regulatable expression element” includes any component of a CRISPR system including but not limited to a Cas9,Cas9 related nucleases, or Cas9-fusion proteins, one or more sgRNA (small guide RNAs), one or more tracrRNAs and/or other polynucleotide feature or motif which imparts regulatable or tunable features to an AAV viral genome encoding them.

Where an AAV polynucleotide encodes one or more CRISPR regulatable expression elements e.g., CRISPR regulatable elements, it is termed herein a “CRISPR-AAV” polynucleotide. CRISPR-AAV polynucleotides may be designed for gene knockdown or for gene replacement (resulting in e.g., activation, initiation, and increased expression).

In some embodiments, the cas9 protein or nuclease is the CRISPR regulatable element and may be selected from any of the known or putative Streptococcus cas9 enzymes in the Uniprot cluster and listed in Table 1. Such proteins, if they are to be delivered as a nucleic acid such as an encoded mRNA and expressed in an organism other than Streptococcus, may be codon optimized for expression in the recipient cell or organism.

TABLE 1
Cas 9 proteins of Streptococcus sp.

| Uniprot | | | SEQ ID |
|---|---|---|---|
| Entry | Protein name | Organism | NO |
| Q03JI6 | CRISPR-associated endonuclease Cas9 | Streptococcus thermophilus (strain | 1 |
| | 2 (EC 3.1.—.—) | ATCC BAA-491/LMD-9) | |
| Q99ZW2 | CRISPR-associated endonuclease | Streptococcus pyogenes serotype M1 | 2 |
| | Cas9/Csn1 (EC 3.1.—.—) | | |
| G3ECR1 | CRISPR-associated endonuclease Cas9 | Streptococcus thermophilus | 3 |
| | (EC 3.1.—.—) | | |
| J3JPT0 | CRISPR-associated protein csn1 | Streptococcus ratti FA-1 = DSM 20564 | 4 |
| | (Uncharacterized protein) | | |
| I5BLK7 | Uncharacterized protein | Streptococcus agalactiae ZQ0910 | 5 |
| K4Q9P5 | Uncharacterized protein | Streptococcus dysgalactiae subsp. | 6 |
| | | equisimilis AC-2713 | |
| Q3D2H4 | Reticulocyte binding protein | Streptococcus agalactiae H36B | 7 |
| M2GS30 | Uncharacterized protein | Streptococcus mutans A19 | 8 |
| Q3DG33 | Reticulocyte binding protein | Streptococcus agalactiae CJB111 | 9 |
| M1YDU0 | Uncharacterized protein | Streptococcus agalactiae LADL-90-503 | 10 |
| M7DS80 | Uncharacterized protein | Streptococcus mutans ATCC 25175 | 11 |
| M7DAQ6 | Uncharacterized protein | Streptococcus mutans KK23 | 12 |
| M2KYB4 | Uncharacterized protein | Streptococcus mutans S1B | 13 |
| I0Q2W2 | CRISPR-associated protein Cas9/Csn1, | Streptococcus oralis SK610 | 14 |
| | subtype II/NMEMI | | |
| Q3DN68 | Reticulocyte binding protein | Streptococcus agalactiae 515 | 15 |
| E9FPR9 | CRISPR-associated protein, Csn1 | Streptococcus sp. M334 | 16 |
| | family | | |
| F5U0T2 | CRISPR-associated protein, Csn1 | Streptococcus anginosus SK52 = DSM | 17 |
| | family | 20563 | |
| H8HE09 | CRISPR-associated protein Csn1 | Streptococcus pyogenes MGAS1882 | 18 |
| M2E8A3 | Uncharacterized protein | Streptococcus mutans 8ID3 | 19 |
| Q3DAP7 | Reticulocyte binding protein | Streptococcus agalactiae COH1 | 20 |
| J7TMY5 | Putative cytosolic protein | Streptococcus salivarius K12 | 21 |
| F2C4I5 | Csn1 family CRISPR-associated protein | Streptococcus sanguinis SK330 | 22 |
| F9HIG7 | CRISPR-associated protein Cas9/Csn1, | Streptococcus sp. oral taxon 056 str. | 23 |
| | subtype II/NMEMI | F0418 | |
| E0PL18 | Csn1 family CRISPR-associated protein | Streptococcus gallolyticus subsp. | 24 |
| | | gallolyticus TX20005 | |
| M4YX12 | CRISPR-associated protein | Streptococcus dysgalactiae subsp. | 25 |
| | | equisimilis RE378 | |
| M1XVB4 | Uncharacterized protein | Streptococcus agalactiae SS1219 | 26 |
| M7E3Z6 | CRISPR-associated protein csn1 | Streptococcus mutans NCTC 11060 | 27 |
| M2ECS5 | Uncharacterized protein | Streptococcus mutans 4SM1 | 28 |
| F0I6Z8 | Csn1 family CRISPR-associated protein | Streptococcus sanguinis SK115 | 29 |
| M2FSD0 | Uncharacterized protein | Streptococcus mutans 2VS1 | 30 |
| J7M7J1 | Uncharacterized protein | Streptococcus pyogenes M1 476 | 31 |
| M2LXP5 | Uncharacterized protein | Streptococcus mutans U2B | 32 |
| M2IJW5 | Uncharacterized protein | Streptococcus mutans M2A | 33 |
| M2KKV5 | Uncharacterized protein | Streptococcus mutans 66-2A | 34 |
| M2IIP5 | Uncharacterized protein | Streptococcus mutans NLML9 | 35 |
| M2DYK8 | Uncharacterized protein | Streptococcus mutans 4VF1 | 36 |
| M2HBR4 | Uncharacterized protein | Streptococcus mutans N66 | 37 |
| G5KAN2 | CRISPR-associated protein Cas9/Csn1, | Streptococcus pseudoporcinus LQ 940- | 38 |
| | subtype II/NMEMI | 04 | |
| M2F746 | Uncharacterized protein | Streptococcus mutans 11VS1 | 39 |
| M2KCP8 | Uncharacterized protein | Streptococcus mutans SA38 | 40 |
| K4N5K1 | CRISPR-associated protein, Csn1 | Streptococcus pyogenes A20 | 41 |
| | family | | |
| M2G9R5 | Uncharacterized protein | Streptococcus mutans A9 | 42 |
| M2KJE3 | Uncharacterized protein | Streptococcus mutans SM4 | 43 |
| M7CZ76 | Uncharacterized protein | Streptococcus mutans KK21 | 44 |
| M2FYT7 | Uncharacterized protein | Streptococcus mutans M21 | 45 |
| F5U4D7 | CRISPR-associated protein, Csn1 | Streptococcus dysgalactiae subsp. | 46 |
| | family (Fragment) | equisimilis SK1249 | |
| F5U5Q4 | Putative uncharacterized protein | Streptococcus dysgalactiae subsp. | 47 |
| | (Fragment) | equisimilis SK1249 | |
| M2H646 | Uncharacterized protein | Streptococcus mutans U138 | 48 |
| M2KHB4 | Uncharacterized protein | Streptococcus mutans SM1 | 49 |
| M2J4V9 | Uncharacterized protein | Streptococcus mutans ST6 | 50 |
| M2F2U6 | Uncharacterized protein | Streptococcus mutans 11SSST2 | 51 |
| G7SP82 | CRISPR-system-like protein | Streptococcus suis ST1 | 52 |
| M2FXA5 | Uncharacterized protein | Streptococcus mutans G123 | 53 |
| M2IJB5 | Uncharacterized protein | Streptococcus mutans NV1996 | 54 |
| M7E6C3 | Uncharacterized protein | Streptococcus mutans AC4446 | 55 |
| K1LK43 | Csn1 family CRISPR-associated protein | Streptococcus iniae 9117 | 56 |
| I0SF74 | CRISPR-associated protein Cas9/Csn1, | Streptococcus constellatus subsp. | 57 |
| | subtype II/NMEMI | constellatus SK53 | |
| M1YIE1 | Uncharacterized protein | Streptococcus agalactiae CF01173 | 58 |
| M2J1X3 | Uncharacterized protein | Streptococcus mutans W6 | 59 |
| I6SW88 | CRISPR-associated protein csn1 | Streptococcus mutans GS-5 | 60 |
| I6Q294 | Csn1 | Streptococcus thermophilus MN-ZLW- | 61 |
| | | 002 | |
| F0FD37 | Csn1 family CRISPR-associated protein | Streptococcus sanguinis SK353 | 62 |
| F9NIK9 | CRISPR-associated protein Cas9/Csn1, | Streptococcus dysgalactiae subsp. | 63 |
| | subtype II/NMEMI | equisimilis SK1250 | |
| F9NIK8 | Putative uncharacterized protein | Streptococcus dysgalactiae subsp. | 64 |
| | | equisimilis SK1250 | |
| M2JCP4 | Uncharacterized protein | Streptococcus mutans B | 65 |
| K4PPI8 | CRISPR-associated protein | Streptococcus agalactiae SA20-06 | 66 |
| E1LI65 | HNH endonuclease family protein | Streptococcus mitis SK321 | 67 |
| E9FJ16 | CRISPR-associated protein, Csn1 | Streptococcus sp. C300 | 68 |
| | family | | |
| M2KGB0 | Uncharacterized protein | Streptococcus mutans 14D | 69 |
| M7DIF2 | CRISPR-associated protein csn1 | Streptococcus mutans 5DC8 | 70 |
| M2INU6 | Uncharacterized protein | Streptococcus mutans SF1 | 71 |
| M2LHR5 | Uncharacterized protein | Streptococcus mutans 24 | 72 |
| J4TM44 | CRISPR-associated protein Cas9/Csn1, | Streptococcus anginosus SK1138 | 73 |
| | subtype II/NMEMI | | |
| M2JLG8 | Uncharacterized protein | Streptococcus mutans SM6 | 74 |
| I7QXF2 | Putative cytoplasmic protein | Streptococcus canis FSL Z3-227 | 75 |
| I3I1V4 | Putative cytoplasmic protein | Streptococcus pyogenes HKU | 76 |
| | | QMH11M0907901 | |
| J8T4Q2 | Uncharacterized protein | Streptococcus agalactiae GB00112 | 77 |
| E7S4M3 | Csn1 family CRISPR-associated protein | Streptococcus agalactiae ATCC 13813 | 78 |
| M2HZK2 | Uncharacterized protein | Streptococcus mutans NLML4 | 79 |
| M2DAT4 | Uncharacterized protein | Streptococcus mutans 1SM1 | 80 |
| M2GPV5 | CRISPR-associated protein | Streptococcus mutans NMT4863 | 81 |
| M5PJI2 | CRISPR-associated protein | Streptococcus parauberis KRS-02109 | 82 |
| F8Y040 | Putative uncharacterized protein | Streptococcus agalactiae FSL S3-026 | 83 |
| M2FXC0 | Uncharacterized protein | Streptococcus mutans 5SM3 | 84 |
| M2ENP9 | Uncharacterized protein | Streptococcus mutans NFSM2 | 85 |
| M1XWD6 | Uncharacterized protein | Streptococcus agalactiae SS1014 | 86 |
| K0U976 | Uncharacterized protein | Streptococcus agalactiae STIR-CD-17 | 87 |
| E0PEL3 | Csn1 family CRISPR-associated protein | Streptococcus bovis ATCC 700338 | 88 |
| M2EUD0 | Uncharacterized protein | Streptococcus mutans 3SN1 | 89 |
| E4L3R1 | CRISPR-associated protein, Csn1 | Streptococcus pseudoporcinus SPIN | 90 |
| | family | 20026 | |
| M2LYU7 | Uncharacterized protein | Streptococcus mutans R221 | 91 |
| M2IAS5 | Uncharacterized protein | Streptococcus mutans N3209 | 92 |
| M2KAP8 | Uncharacterized protein | Streptococcus mutans NLML1 | 93 |
| M2F4E1 | Uncharacterized protein | Streptococcus mutans N29 | 94 |
| E8JP81 | Csn1 family CRISPR-associated protein | Streptococcus equinus ATCC 9812 | 95 |
| M2G1L7 | Uncharacterized protein | Streptococcus mutans NVAB | 96 |
| J4K985 | CRISPR-associated protein Cas9/Csn1, | Streptococcus oralis SK304 | 97 |
| | subtype II/NMEMI | | |
| M2E7C1 | CRISPR-associated protein | Streptococcus mutans 15VF2 | 98 |
| Q3DQT5 | CRISPR-associated protein, SAG0894 | Streptococcus agalactiae 18RS21 | 99 |
| | family (Fragment) | | |
| M2IP01 | Uncharacterized protein | Streptococcus mutans SF14 | 100 |
| E6J3R0 | CRISPR-associated protein, Csn1 | Streptococcus anginosus F0211 | 101 |
| | family | | |
| G5JVJ9 | CRISPR-associated protein Cas9/Csn1, | Streptococcus macacae NCTC 11558 | 102 |
| | subtype II/NMEMI | | |
| K8MQ90 | CRISPR-associated protein cas9/csn1, | Streptococcus sp. F0441 | 103 |
| | subtype II/nmemi | | |
| M2KYT3 | Uncharacterized protein | Streptococcus mutans M230 | 104 |
| H8HAK7 | CRISPR-associated protein Csn1 | Streptococcus pyogenes MGAS15252 | 105 |
| M2HZJ5 | Uncharacterized protein | Streptococcus mutans NFSM1 | 106 |
| E4SQY2 | CRISPR-associated endonuclease, Csn1 | Streptococcus thermophilus (strain | 107 |
| | family | ND03) | |
| Q1JC13 | Hypothetical cytosolic protein | Streptococcus pyogenes serotype M12 | 108 |
| | | (strain MGAS2096) | |
| C6SPS8 | Uncharacterized protein | Streptococcus mutans serotype c (strain | 109 |
| | | NN2025) | |
| C5WH61 | CRISPR-associated protein Csn1 | Streptococcus dysgalactiae subsp. | 110 |
| | | equisimilis (strain GGS 124) | |
| Q8E042 | Putative uncharacterized protein | Streptococcus agalactiae serotype V | 111 |
| | | (strain ATCC BAA-611/2603 V/R) | |
| F5WVJ4 | CRISPR-associated protein | Streptococcus gallolyticus (strain ATCC | 112 |
| | | 43143/F-1867) | |
| D3HEH4 | CRISPR-associated protein | Streptococcus gallolyticus (strain | 113 |
| | | UCN34) | |
| J9YP56 | Uncharacterized protein | Streptococcus agalactiae serotype Ia | 114 |
| | | (strain GD201008-001) | |
| Q8E5R9 | Putative uncharacterized protein | Streptococcus agalactiae serotype III | 115 |
| | gbs0911 | (strain NEM316) | |
| F7IUC8 | Putative uncharacterized protein | Streptococcus pyogenes serotype M3 | 116 |
| | SPs1176 | (strain SSI-1) | |
| Q8DTE3 | Putative uncharacterized protein | Streptococcus mutans serotype c (strain | 117 |
| | | ATCC 700610/UA159) | |
| F0VS85 | CRISPR-associated protein | Streptococcus gallolyticus (strain ATCC | 118 |
| | | BAA-2069) | |
| B5XLC1 | Putative uncharacterized protein | Streptococcus pyogenes serotype M49 | 119 |
| | | (strain NZ131) | |
| Q3K1G4 | CRISPR-associated protein, SAG0894 | Streptococcus agalactiae serotype Ia | 120 |
| | family | (strain ATCC 27591/A909/CDC | |
| | | SS700) | |
| E8QAX4 | Hypothetical cytosolic protein | Streptococcus dysgalactiae subsp. | 121 |
| | | equisimilis (strain ATCC 12394/ | |
| | | D166B) | |
| H6PBR9 | CRISPR-associated protein, SAG0894 | Streptococcus infantarius (strain CJ18) | 122 |
| | family | | |
| Q48Z31 | Hypothetical cytosolic protein | Streptococcus pyogenes serotype M1 | 123 |
| Q1J6W2 | Hypothetical cytosolic protein | Streptococcus pyogenes serotype M4 | 124 |
| | | (strain MGAS10750) | |
| Q8K7R2 | Putative uncharacterized protein | Streptococcus pyogenes serotype M3 | 125 |
| | | (strain ATCC BAA-595/MGAS315) | |
| Q1JLZ6 | Hypothetical cytosolic protein | Streptococcus pyogenes serotype M12 | 126 |
| | | (strain MGAS9429) | |
| Q48TU5 | Hypothetical cytosolic protein | Streptococcus pyogenes serotype M28 | 127 |
| | | (strain MGAS6180) | |
| Q1JH43 | Hypothetical cytosolic protein | Streptococcus pyogenes serotype M2 | 128 |
| | | (strain MGAS10270) | |

Chylinski et al. (RNA Biology, 2013, 10:5, 726-737, herein incorporated by reference in its entirety including supplemental materials), has identified several Cas9 orthologs from Type II CRISPR-Cas loci.

According to the present invention, these enzymes may also serve as genome editing enzymes, e.g., CRISPR regulatable elements, of the invention and are given in Table 2. Given in the table are the gi accession numbers from NCBI and the name of the bacterial strain. It will be understood that such enzymes, when expressed in any organism other than the wild type strain may be codon optimized for that organism at the nucleic acid level.

TABLE 2
Cas9 Orthologs

| | | SEQ ID |
|---|---|---|
| gi Number | Strain | NO |
| 491523080 | Veillonella atypica ACS-134-V-Col7a | 129 |
| 492568239 | Fusobacterium nucleatum subsp. vincentii | 130 |
| | ATCC 49256 | |
| 291166249 | Filifactor alocis ATCC 35896 | 131 |
| 320130861 | Solobacterium moorei F0204 | 132 |
| 291520705 | Coprococcus catus GD-7 | 133 |
| 42525843 | Treponema denticola ATCC 35405 | 134 |
| 496176552 | Peptoniphilus duerdenii ATCC BAA-1640 | 135 |
| 493553119 | Catenibacterium mitsuokai DSM 15897 | 136 |
| 24379809 | Streptococcus mutans UA159 | 137 |
| 15675041 | Streptococcus pyogenes SF370 | 138 |
| 499300419 | Listeria innocua Clip11262 | 139 |
| 500000752 | Streptococcus thermophilus LMD-9 | 140 |
| 323463801 | Staphylococcus pseudintermedius | 141 |
| 352684361 | Acidaminococcus intestini RyC-MR95 | 142 |
| 503017123 | Olsenella uli DSM 7084 | 143 |
| 366983953 | Oenococcus kitaharae DSM 17330 | 144 |
| 503128334 | Bifidobacterium bifidum S17 | 145 |
| 504382875 | Lactobacillus rhamnosus GG | 146 |
| 489744644 | Lactobacillus gasseri JV-V03 | 147 |
| 501247123 | Finegoldia magna ATCC 29328 | 148 |
| 47458196 | Mycoplasma mobile 163K | 149 |
| 284931710 | Mycoplasma gallisepticum str. F | 150 |
| 498006766 | Mycoplasma ovipneumoniae SC01 | 151 |
| 384393286 | Mycoplasma canis PG 14 | 152 |
| 144575181 | Mycoplasma synoviae 53 | 153 |
| 238875750 | Eubacterium rectale ATCC 33656 | 154 |
| 500000239 | Streptococcus thermophilus LMD-9 | 155 |
| 315149830 | Enterococcus faecalis TX0012 | 156 |
| 488391463 | Staphylococcus lugdunensis M23590 | 157 |
| 158432258 | Eubacterium dolichum DSM 3991 | 158 |
| 497700222 | Lactobacillus coryniformis subsp. torquens | 159 |
| | KCTC 3535 | |
| 503154365 | Ilyobacter polytropus DSM 2926 | 160 |
| 488935851 | Ruminococcus albus 8 | 161 |
| 187426541 | Akkermansia muciniphila ATCC BAA-835 | 162 |
| 117649621 | Acidothermus cellulolyticus 11B | 163 |
| 189429199 | Bifidobacterium longum DJO10A | 164 |
| 502666262 | Bifidobacterium dentium Bd1 | 165 |
| 499236428 | Corynebacterium diphtheriae NCTC 13129 | 166 |
| 501382854 | Elusimicrobium minutum Pei191 | 167 |
| 319419610 | Nitratifractor salsuginis DSM 16511 | 168 |
| 324027241 | Sphaerochaeta globus str. Buddy | 169 |
| 502574305 | Fibrobacter succinogenes subsp. succinogenes | 170 |
| | S85 | |
| 496648031 | Bacteroides fragilis NCTC 9343 | 171 |
| 506262077 | Capnocytophaga ochracea DSM 7271 | 172 |
| 499794158 | Rhodopseudomonas palustris BisB18 | 173 |
| 494010777 | Prevotella micans F0438 | 174 |
| 294472455 | Prevotella ruminicola 23 | 175 |
| 503930464 | Flavobacterium columnare ATCC 49512 | 176 |
| 310782306 | Aminomonas paucivorans DSM 12260 | 177 |
| 83591793 | Rhodospirillum rubrum ATCC 11170 | 178 |
| 502812437 | Candidatus Puniceispirillum marinum | 179 |
| | IMCC1322 | |
| 500133006 | Verminephrobacter eiseniae EF01-2 | 180 |
| 344171927 | Ralstonia syzygii R24 | 181 |
| 159042956 | Dinoroseobacter shibae DFL 12 | 182 |
| 288910049 | Azospirillum sp-B510 | 183 |
| 91802344 | Nitrobacter hamburgensis X14 | 184 |
| 146407516 | Bradyrhizobium sp-BTAi1 | 185 |
| 499451825 | Wolinella succinogenes DSM 1740 | 186 |
| 218563121 | Campylobacter jejuni subsp. jejuni NCTC 11168 | 187 |
| 502787413 | Helicobacter mustelae 12198 | 188 |
| 447027826 | Bacillus cereus Rock1-15 | 189 |
| 501844634 | Acidovorax ebreus TPSY | 190 |
| 189485225 | uncultured Termite group 1 bacterium | 191 |
| | phylotype RsD17 | |
| 489569047 | Clostridium perfringens D str. JGS1721 | 192 |
| 506406750 | Clostridium cellulolyticum H10 | 193 |
| 154154505 | Parvibaculum lavamentivorans DS-1 | 194 |
| 493910016 | Roseburia intestinalis L1-82 | 195 |
| 488163954 | Neisseria meningitidis Z2491 | 196 |
| 499209493 | Pasteurella multocida subsp. multocida str. Pm70 | 197 |
| 491573077 | Sutterella wadsworthensis 3 1 45B | 198 |
| 495559660 | gamma proteobacterium HTCC5015 | 199 |
| 499526152 | Legionella pneumophila str. Paris | 200 |
| 496140336 | Parasutterella excrementihominis YIT 11859 | 201 |
| 499451967 | Wolinella succinogenes DSM 1740 | 202 |
| 489129153 | Francisella novicida U112 | 203 |

Any of the enzymes or proteins of Tables 1 or 2 may be a CRISPR regulatable element. Further, any cas9 or cas9 ortholog may be fused to a destabilizing domain such as, but not limited to, FK506 Binding Protein (FKBP), E. coli dihydrofolate reductase (DHFR), mouse ornithine decarboxylase (MODC), or estrogen receptors (ER).

In one embodiment, the destabilizing domain may be from an estrogen receptor.

In one embodiment, the CRISPR regulatable element may comprise a destabilizing sequence such as, but not limited to, a 3′UTR with AU-rich elements or AUUUA motifs. Destabilizing sequences can be chosen from those in cytokines, proto-oncogenes, interferon mRNAs or human estrogen receptor alpha. As a non-limiting example, the mRNA sequence for any cas9 or cas9 ortholog may comprise a destabilizing sequence, such as, but not limited to, a 3′UTR with AU-rich elements or AUUUA motifs.

Several protospacer adjacent motifs (PAMs) have been identified in the art (Westra et al., Annu. Rev. Genet. 2012, 46: 311-39; the contents of which are incorporated herein by reference in their entirety). Such PAMs may be used to inform the selection of and/or design of the nucleic acid compositions, e.g., CRISPR-AAV constructs, of the present invention. It is also contemplated that utilization of certain Type III enzymes such as those from Staphylococcus epidermidis, Pyrococcus furiosus or S. solfatarcicus will not require the presence of a PAM sequence. PAMs useful in the present invention are given in Table 3. In the table the PAM either follows or precedes the protospacer (that region of the DNA found immediately upstream or downstream of the PAM and on the opposite DNA strand that hybridizes with the sgRNA).

TABLE 3
PAM sequences

| PAM | SEQ ID NO |
|---|---|
| Protospacer-NGG | 204-207 |
| Protospacer-GAA | 208-209 |
| Protospacer-CTT | 2010-211 |
| Protospacer-CAT | 212 |
| Protospacer-CCT | 213 |
| Protospacer-CTC | 214 |
| Protospacer-GG | 215-216 |
| WTTCTNN-Protospacer | 217 |
| TTTYRNNN-Protospacer | 218 |
| CNCCN-Protospacer | 219 |
| CCN-Protospacer | 220-21 |

According to the present invention, AAV particles comprising the nucleic acids of the CRISPR regulatable elements may be AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rh.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, and/or AAVrh.74 serotypes, and variants thereof.

In one embodiment, the AAV serotype may be or have a sequence as described in United States Publication No. US20030138772, herein incorporated by reference in its entirety, such as, but not limited to, AAV1 (SEQ ID NO: 6 and 64 of US20030138772), AAV2 (SEQ ID NO: 7 and 70 of US20030138772), AAV3 (SEQ ID NO: 8 and 71 of US20030138772), AAV4 (SEQ ID NO: 63 of US20030138772), AAV5 (SEQ ID NO: 114 of US20030138772), AAV6 (SEQ ID NO: 65 of US20030138772), AAV7 (SEQ ID NO: 1-3 of US20030138772), AAV8 (SEQ ID NO: 4 and 95 of US20030138772), AAV9 (SEQ ID NO: 5 and 100 of US20030138772), AAV10 (SEQ ID NO: 117 of US20030138772), AAV11 (SEQ ID NO: 118 of US20030138772), AAV12 (SEQ ID NO: 119 of US20030138772), AAVrh10 (amino acids 1 to 738 of SEQ ID NO: 81 of US20030138772) or variants thereof. Non limiting examples of variants include SEQ ID NOs: 9, 27-45, 47-62, 66-69, 73-81, 84-94, 96, 97, 99, 101-113 of US20030138772, the contents of which are herein incorporated by reference in its entirety.

In one embodiment, the AAV serotype may be or may have a sequence as described in United States Publication No. US20150159173, herein incorporated by reference in its entirety, such as, but not limited to, AAV2 (SEQ ID NO: 7 and 23 of US20150159173), rh20 (SEQ ID NO: 1 of US20150159173), rh32/33 (SEQ ID NO: 2 of US20150159173), rh39 (SEQ ID NO: 3, 20 and 36 of US20150159173), rh46 (SEQ ID NO: 4 and 22 of US20150159173), rh73 (SEQ ID NO: 5 of US20150159173), rh74 (SEQ ID NO: 6 of US20150159173), AAV6.1 (SEQ ID NO: 29 of US20150159173), rh.8 (SEQ ID NO: 41 of US20150159173), rh.48.1 (SEQ ID NO: 44 of US20150159173), hu.44 (SEQ ID NO: 45 of US20150159173), hu.29 (SEQ ID NO: 42 of US20150159173), hu.48 (SEQ ID NO: 38 of US20150159173), rh54 (SEQ ID NO: 49 of US20150159173), AAV2 (SEQ ID NO: 7 of US20150159173), cy.5 (SEQ ID NO: 8 and 24 of US20150159173), rh.10 (SEQ ID NO: 9 and 25 of US20150159173), rh.13 (SEQ ID NO: 10 and 26 of US20150159173), AAV1 (SEQ ID NO: 11 and 27 of US20150159173), AAV3 (SEQ ID NO: 12 and 28 of US20150159173), AAV6 (SEQ ID NO: 13 and 29 of US20150159173), AAV7 (SEQ ID NO: 14 and 30 of US20150159173), AAV8 (SEQ ID NO: 15 and 31 of US20150159173), hu.13 (SEQ ID NO: 16 and 32 of US20150159173), hu.26 (SEQ ID NO: 17 and 33 of US20150159173), hu.37 (SEQ ID NO: 18 and 34 of US20150159173), hu.53 (SEQ ID NO: 19 and 35 of US20150159173), rh.43 (SEQ ID NO: 21 and 37 of US20150159173), rh2 (SEQ ID NO: 39 of US20150159173), rh.37 (SEQ ID NO: 40 of US20150159173), rh.64 (SEQ ID NO: 43 of US20150159173), rh.48 (SEQ ID NO: 44 of US20150159173), ch.5 (SEQ ID NO 46 of US20150159173), rh.67 (SEQ ID NO: 47 of US20150159173), rh.58 (SEQ ID NO: 48 of US20150159173), or variants thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In one embodiment, the AAV serotype may be or have the sequence as described in U.S. Pat. No. 7,198,951, herein incorporated by reference in its entirety, such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), and AAV8 (SEQ ID NO: 7).

In one embodiment, the AAV serotype may be or have a mutation in the AAV9 sequence as described by N Pulicherla et al. (Molcular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In one embodiment, the AAV serotype may be or have a sequence as described in U.S. Pat. No. 6,156,303, herein incorporated by reference in its entirety, such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or derivatives thereof.

In one embodiment, the AAV serotype may be or may have a sequence as described in United States Publication No. US20140359799, herein incorporated by reference in its entirety, such as, but not limited to, AAV8 (SEQ ID NO: 1 of US20140359799), AAVDJ (SEQ ID NO: 2 and 3 of US20140359799), or variants thereof.

In one embodiment, the AAV serotype may be or have the sequence of AAV4 as described in International Publication No. WO1998011244, herein incorporated by reference in its entirety, such as, but not limited to AAV4 (SEQ ID NO: 1-20 of WO1998011244).

In one embodiment, the AAV serotype may be or have a mutation in the AAV2 sequence to generate AAV2G9 as described in International Publication No. WO2014144229 and herein incorporated by reference in its entirety.

In one embodiment, the AAV serotype may be or have a sequence as described in International Publication WO2005033321, herein incorporated by reference in its entirety, such as, but not limited to AAV1 (SEQ ID NO: 202 and 219 of WO2005033321), AAV2 (SEQ ID NO: 211 and 221 of WO2005033321), AAV3-3 (SEQ ID NO: 200 and 217 of WO2005033321), AAV4-4 (SEQ ID NO: 201 and 218 of WO2005033321), AAV5 (SEQ ID NO: 216 and 199 of WO2005033321), AAV6 (SEQ ID NO: 203 and 220 of WO2005033321), AAV7 (SEQ ID NO: 213 and 222 of WO2005033321), AAV8 (SEQ ID NO: 214 and 223 of WO2005033321), hu.14/AAV9 (SEQ ID NO: 3 and 123 of WO2005033321), hu.17 (SEQ ID NO: 83 of WO2005033321), hu.6 (SEQ ID NO: 84 of WO2005033321), hu.42 (SEQ ID NO: 85 of WO2005033321), rh.38 (SEQ ID NO: 86 of WO2005033321), hu.40 (SEQ ID NO: 87 of WO2005033321), hu.37 (SEQ ID NO: 88 of WO2005033321), rh.40 (SEQ ID NO: 92 of WO2005033321), rh.52 (SEQ ID NO: 96 of WO2005033321), rh.53 (SEQ ID NO: 97 of WO2005033321), rh.49 (SEQ ID NO: 103 of WO2005033321), rh.51 (SEQ ID NO: 104 of WO2005033321), rh.57 (SEQ ID NO: 105 of WO2005033321), rh.58 (SEQ ID NO: 106 of WO2005033321), rh.61 (SEQ ID NO: 107 of WO2005033321), rh.50 (SEQ ID NO: 108 of WO2005033321), rh.43 (SEQ ID NO: 163 of WO2005033321), rh.62 (SEQ ID NO: 114 of WO2005033321), rh.48 (SEQ ID NO: 115 of WO2005033321), 4-9/rh.54 (SEQ ID NO: 116 of WO2005033321), 4-19/rh.55 (SEQ ID NO: 117 of WO2005033321), hu.31 (SEQ ID NO: 121 of WO2005033321), hu.32 (SEQ ID NO: 122 of WO2005033321), hu.34 (SEQ ID NO: 125 of WO2005033321), hu.45 (SEQ ID NO: 127 of WO2005033321), hu.47 (SEQ ID NO: 128 of WO2005033321), hu.13 (SEQ ID NO: 129 of WO2005033321), hu.28 (SEQ ID NO: 130 of WO2005033321), hu.29 (SEQ ID NO: 132 of WO2005033321), hu.19 (SEQ ID NO: 133 of WO2005033321), hu.20 (SEQ ID NO: 134 of WO2005033321), hu.21 (SEQ ID NO: 135 of WO2005033321), hu.23.2 (SEQ ID NO: 137 of WO2005033321), hu.22 (SEQ ID NO: 138 of WO2005033321), hu.27 (SEQ ID NO: 140 of WO2005033321), hu.4 (SEQ ID NO: 141 of WO2005033321), hu.2 (SEQ ID NO: 143 of WO2005033321), hu.1 (SEQ ID NO: 144 of WO2005033321), hu.3 (SEQ ID NO: 145 of WO2005033321), hu.25 (SEQ ID NO: 146 of WO2005033321), hu.15 (SEQ ID NO: 147 of WO2005033321), hu.16 (SEQ ID NO: 148 of WO2005033321), hu.18 (SEQ ID NO: 149 of WO2005033321), hu.7 (SEQ ID NO: 150 of WO2005033321), hu.11 (SEQ ID NO: 153 of WO2005033321), hu.9 (SEQ ID NO: 155 of WO2005033321), hu.10 (SEQ ID NO: 156 of WO2005033321), hu.48 (SEQ ID NO: 157 of WO2005033321), hu.44 (SEQ ID NO: 144 of WO2005033321), hu.46 (SEQ ID NO: 159 of WO2005033321), hu.43 (SEQ ID NO: 160 of WO2005033321), hu.35 (SEQ ID NO: 164 of WO2005033321), hu.24 (SEQ ID NO: 136 of WO2005033321), rh.64 (SEQ ID NO: 99 of WO2005033321), hu.41 (SEQ ID NO: 91 of WO2005033321), hu.39 (SEQ ID NO: 102 of WO2005033321), hu.67 (SEQ ID NO: 198 of WO2005033321), hu.66 (SEQ ID NO: 197 of WO2005033321), hu.51 (SEQ ID NO: 190 of WO2005033321), hu.52 (SEQ ID NO: 191 of WO2005033321), hu.49 (SEQ ID NO: 189 of WO2005033321), hu.56 (SEQ ID NO: 192 of WO2005033321), hu.57 (SEQ ID NO: 193 of WO2005033321), hu.58 (SEQ ID NO: 194 of WO2005033321), hu.63 (SEQ ID NO: 195 of WO2005033321), hu.64 (SEQ ID NO: 196), hu.60 (SEQ ID NO: 184 of WO2005033321), hu.61 (SEQ ID NO: 185 of WO2005033321), hu.53 (SEQ ID NO: 186 of WO2005033321), hu.55 (SEQ ID NO: 187 of WO2005033321), hu.54 (SEQ ID NO: 188 of WO2005033321), hu.6 (SEQ ID NO: 84 of WO2005033321), rh.56 (SEQ ID NO: 152 of WO2005033321), or variants thereof. Non limiting examples of variants include SEQ ID NOs: 1, 2, 4-82, 89, 90, 93-95, 98, 100, 101, 109-113, 118-120, 124, 126, 131, 139, 142, 151, 154, 158, 161, 162, 165-183, 202, 204-212, 215, 219, 224-236 of WO2005033321, the contents of which are herein incorporated by reference in its entirety.

In one embodiment, the serotype which may be useful in the present invention may be AAVDJ8 (or AAV-DJ8). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

The present invention also provides pharmaceutical compositions comprising at least one AAV particle having a CRISPR regulatable element and a pharmaceutically acceptable excipient. In some aspects, one or more CRISPR regulatable elements are contained in an AAV particle.

CRISPR-AAV Particle Payload: Transgenes, Polypeptide-Encoding Polynucleotides. and/or Modulatory Nucleic Acids

In some embodiments, the present invention provides methods for inhibiting, silencing or inducing, activating, and/or initiating expression of a gene in a cell.

The CRISPR-AAV particles of the present invention comprise a nucleic acid sequence e.g., polynucleotide, encoding at least one “payload.” As used herein, a “payload” refers to one or more polynucleotides or polynucleotide regions encoded by or within a viral genome or an expression product of such polynucleotide or polynucleotide region e.g., a transgene, a polynucleotide encoding a polypeptide or multi polypeptide or a modulatory nucleic acid or regulatory nucleic acid.

The payload may comprise any nucleic acid known in the art which is useful for modulating the expression (by supplementation or gene replacement or by inhibition using a modulatory nucleic acid) in a target cell transduced or contacted with the CRISPR-AAV particle carrying the payload.

Vectors used in the production of CRISPR-AAV particles include those encoding the payload, e.g. payload construct vectors, and those encoding accessory proteins necessary for production, e.g. viral construct vectors.

According to the present invention, a CRISPR-AAV payload construct vector encodes a “payload construct.” In one embodiment, a “payload construct” is a polynucleotide sequence encoding at least a payload and sufficient ITR sequence to allow for replication thereby producing a viral genome that is packaged inside a capsid.

The payload construct may comprise a combination of coding and non-coding nucleic acid sequences.

In one embodiment, the CRISPR-AAV payload construct vector comprises more than one nucleic acid sequences encoding more than one payload of interest. In such an embodiment, a payload construct encoding more than one payload may be replicated and packaged into a viral particle. A target cell transduced with a viral particle comprising more than one payload may express each of the payloads in a single cell.

In some embodiments, the CRISPR-AAV payload construct may encode a coding or non-coding RNA.

Where the CRISPR-AAV payload construct sequence encodes a polypeptide, the polypeptide may be a peptide or protein. A protein encoded by the CRISPR-AAV payload construct sequence may comprise a secreted protein, an intracellular protein, an extracellular protein, and/or a membrane protein. The encoded proteins may be structural or functional. Proteins encoded by the payload construct vector or payload construct include, but are not limited to, mammalian proteins. The CRISPR-AAV polynucleotides encoding polypeptides (e.g., mRNA) of the invention may be useful in the fields of human disease, antibodies, viruses, veterinary applications and a variety of in vivo and in vitro settings.

In some embodiments, the CRISPR-AAV particles are useful in the field of medicine for the treatment, palliation or amelioration of conditions or diseases such as, but not limited to, blood, cardiovascular, CNS, dermatology, endocrinology, genetic, genitourinary, gastrointestinal, musculoskeletal, oncology, and immunology, respiratory, sensory and anti-infective.

In some embodiments, CRISPR-AAV particles in accordance with the present invention may be used for the treatment of disorders, and/or conditions, including but not limited to one or more of the following: autoimmune disorders (e.g. diabetes, lupus, multiple sclerosis, psoriasis, rheumatoid arthritis); inflammatory disorders (e.g. arthritis, pelvic inflammatory disease); neurological disorders (e.g. Alzheimer's disease, Huntington's disease; autism; Parkinson's disease, amyotrophic lateral sclerosis, Friedrich's Ataxia, spinal muscular atrophy); cardiovascular disorders (e.g. atherosclerosis, hypercholesterolemia, thrombosis, clotting disorders, angiogenic disorders such as macular degeneration); proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia); etc.

In some embodiments, the CRISPR-AAV payload construct encodes a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo.

Traditionally, the basic components of an mRNA molecule include at least a coding region, a 5′UTR, a 3′UTR, a 5′ cap and a poly-A tail. According to the present invention, CRISPR-AAV payload constructs encoding mRNA may comprise a coding region only. They may also comprise a coding region and at least one UTR. They may also comprise a coding region, 3′UTR and polyA tail. In some embodiments, the mRNA or any portion of the CRISPR-AAV may be codon optimized.

In one embodiment the polypeptide encoded by the payload construct is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-1000 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length.

In some embodiments the peptide encoded by the payload construct is between 4-50 amino acids in length. In one embodiment, the shortest length of a region of the payload molecule of the present invention encoding a peptide can be the length that is sufficient to encode for a tetrapeptide, a pentapeptide, a hexapeptide, a heptapeptide, an octapeptide, a nonapeptide, or a decapeptide. In another embodiment, the length may be sufficient to encode a peptide of 2-30 amino acids, e.g. 5-30, 10-30, 2-25, 5-25, 10-25, or 10-20 amino acids. The length may be sufficient to encode for a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

Modulatory Nucleic Acids

An RNA encoded by the CRISPR-AAV payload construct may comprise an mRNA, tRNA, rRNA, tmRNA, miRNA, RNAi, siRNA, piRNA, shRNA antisense RNA, double stranded RNA, snRNA, snoRNA, and long non-coding RNA (ncRNA). Examples of such lncRNA molecules and RNAi constructs designed to target such lncRNA any of which may be encoded in the payload constructs are taught in International Publication WO2012/018881, the contents of which are incorporated herein by reference in their entirety.

In one embodiment, the CRISPR-AAV payload construct encodes a microRNA or miRNA or engineered precursors thereof as the payload. These payloads along with siRNA, shRNA antisense molecules and the like are also referred to as modulatory nucleic acid payloads.

microRNAs (or miRNA) are 19-25 nucleotide long noncoding RNAs that bind to the 3′UTR of nucleic acid molecules and down-regulate gene expression either by reducing nucleic acid molecule stability or by inhibiting translation. The payload constructs of the invention may comprise one or more microRNA target sequences, microRNA sequences, or microRNA seeds. Such sequences may correspond to any known microRNA such as those taught in US Publication US2005/0261218 and US Publication US2005/0059005, the contents of which are incorporated herein by reference in their entirety.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol Cell. 2007 Jul. 6; 27(1):91-105; each of which is herein incorporated by reference in their entirety. The bases of the microRNA seed have complete complementarity with the target sequence.

### Functional Payloads

A payload may comprise polypeptides that serve as marker proteins to assess cell transformation and expression, fusion proteins, polypeptides having a desired biological activity, gene products that can complement a genetic defect, RNA molecules, transcription factors, and other gene products that are of interest in regulation and/or expression. A payload may comprise nucleotide sequences that provide a desired effect or regulatory function (e.g., transposons, transcription factors). A payload may comprise hormone receptors (e.g., mineral corticosteroid, glucocorticoid, and thyroid hormone receptors); intramembrane proteins (e.g., TM-1 and TM-7); intracellular receptors (e.g., orphans, retinoids, vitamin D3 and vitamin A receptors); signaling molecules (e.g., kinases, transcription factors, or molecules such signal transducers and activators of transcription receptors of the cytokine superfamily (e.g. erythropoietin, growth hormone, interferons, and interleukins, and colony-stimulating factors; G-protein coupled receptors, e.g., hormones, calcitonin, epinephrine, gastrin, and paracrine or autocrine mediators, such as somatostatin or prostaglandins; neurotransmitter receptors (norepinephrine, dopamine, serotonin or acetylcholine); pathogenic antigens, which can be of viral, bacterial, allergenic, or cancerous origin; and tyrosine kinase receptors (such as insulin growth factor, and nerve growth factor).

A payload may comprise a gene therapy product. A gene therapy product may comprise a polypeptide, RNA molecule, or other gene product that, when expressed in a target cell, provides a desired therapeutic effect. In some embodiments, a gene therapy product may comprise a substitute for a non-functional gene that is absent or mutated.

A CRISPR-AAV payload construct encoding a payload may comprise a selectable marker. A selectable marker may comprise a gene sequence or a protein encoded by that gene sequence expressed in a host cell that allows for the identification, selection, and/or purification of the host cell from a population of cells that may or may not express the selectable marker. In one embodiment the selectable marker provides resistance to survive a selection process that would otherwise kill the host cell, such as treatment with an antibiotic. In some embodiments an antibiotic selectable marker may comprise one or more antibiotic resistance factors, including but not limited to neomycin resistance (e.g., neo), hygromycin resistance, kanamycin resistance, and/or puromycin resistance.

In some embodiments a selectable marker may comprise a cell-surface marker, such as any protein expressed on the surface of the cell including, but not limited to receptors, CD markers, lectins, integrins, or truncated versions thereof. In some embodiments, cells that comprise a cell-surface marker may be selected using an antibody targeted to the cell-surface marker. In some embodiments an antibody targeted to the cell-surface marker may be directly conjugated with a selection agent including, but not limited to a fluorophore, sepharose, or magnetic bead. In some embodiments an antibody targeted to the cell-surface marker may be detected using a secondary labeled antibody or substrate which binds to the antibody targeted to the cell-surface marker. In some embodiments, a selectable marker may comprise negative selection by using an enzyme, including but not limited to Herpes simplex virus thymidine kinase (HSVTK) that converts a pro-toxin (ganciclovir) into a toxin or bacterial Cytosine Deaminase (CD) which converts the pro-toxin 5′-fluorocytosine (5′-FC) into the toxin 5′-fluorouracil (5′-FU). In some embodiments, any nucleic acid sequence encoding a polypeptide can be used as a selectable marker comprising recognition by a specific antibody.

In some embodiments, a payload construct encoding a payload may comprise a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO 96/23810; Heim et al., Current Biology 2:178-182 (1996); Heim et al., Proc. Natl. Acad. Sci. USA (1995); or Heim et al., Science 373:663-664 (1995); for β-lactamase, see WO 96/30540). In some embodiments, a nucleic acid encoding a selectable marker may comprise a fluorescent protein. A fluorescent protein as herein described may comprise any fluorescent marker including but not limited to green, yellow, and/or red fluorescent protein (GFP, YFP, and/or RFP).

In some embodiments, a payload construct encoding a payload may comprise a CRISPR regulatable element within the open reading frame in addition to the payload. As a non-limiting example, the CRISPR regulatable element may be a DNA binding domain, a transactivating factor for cas9 or a DNA binding domain coupled to a transactivating factor for cas9. The DNA binding domain may be coupled to the transactivating factor using any methods known in the art or described herein.

In one embodiment the DNA binding domain for the cas9 promoter and/or a transactivating factor for cas9 may be located within the sequence encoding the VP2 capsid. As a non-limiting example, the promoter and/or transactiving factor may be located within the first 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. As another non-limiting example, the promoter and/or transactiving factor may be located within the last 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. As a non-limiting example, the promoter and/or transactivating factor may be located in the middle of the VP2 capsid. As another non-limiting example, the promoter and/or transactiving factor may be located near the beginning of the VP2 capsid. As yet another non-limiting example, the promoter and/or transactiving factor may be located near the end of the VP2 capsid.

In accordance with the invention, a payload comprising a nucleic acid for expression in a target cell will be incorporated into the viral particle produced in the viral replication cell where the payload is located between two ITR sequences, or is located on either side of an asymmetrical ITR engineered with two D regions.

A CRISPR-AAV payload construct encoding one or more payloads for expression in a target cell may comprise one or more payload or non-payload nucleotide sequences operably linked to at least one target cell-compatible promoter. A person skilled in the art may recognize that a target cell may require a specific promoter including but not limited to a promoter that is species specific, inducible, tissue-specific, or cell cycle-specific Parr et al., Nat. Med. 3:1145-9 (1997).

### Viral Production

The present disclosure provides a method for the generation of parvoviral particles, e.g. CRISPR-AAV particles, by viral genome replication in a viral replication cell comprising contacting the viral replication cell with an AAV polynucleotide or AAV genome.

The present disclosure provides a method for producing a CRISPR-AAV particle having enhanced (increased, improved) transduction efficiency comprising the steps of: 1) co-transfecting competent bacterial cells with a bacmid vector and either a viral construct vector and/or CRISPR-AAV payload construct vector, 2) isolating the resultant viral construct expression vector and CRISPR-AAV payload construct expression vector and separately transfecting viral replication cells, 3) isolating and purifying resultant payload and viral construct particles comprising viral construct expression vector or CRISPR-AAV payload construct expression vector, 4) co-infecting a viral replication cell with both the CRISPR-AAV payload and viral construct particles comprising viral construct expression vector or CRISPR-AAV payload construct expression vector, 5) harvesting and purifying the viral particle comprising a CRISPR-parvoviral genome.

### Vectors and Capsids

The invention also provides nucleic acids encoding the mutated or modified virus capsids and capsid proteins of the invention. In some embodiments the capsids are engineered according to the methods of co-owned and co-pending application U.S. 62/009,430 filed Jun. 9, 2014, the contents of which are incorporated herein by reference in their entirety and methods known in the art.

Further provided are vectors comprising the nucleic acids, and cells (in vivo or in culture) comprising the nucleic acids and/or vectors of the invention. Suitable vectors include without limitation viral vectors (e.g., adenovirus, AAV, herpes virus, vaccinia, poxviruses, baculoviruses, and the like), plasmids, phage, YACs, BACs, and the like as are well known in the art. Such nucleic acids, vectors and cells can be used, for example, as reagents (e.g., helper packaging constructs or packaging cells) for the production of modified virus capsids or virus vectors as described herein.

The molecules of the invention which contain AAV sequences include any genetic element (vector) which may be delivered to a host cell, e.g., naked DNA, a plasmid, phage, transposon, cosmid, episome, a protein in a non-viral delivery vehicle (e.g., a lipid-based carrier), virus, etc., which transfers the sequences carried thereon. The selected vector may be delivered by any suitable method, including transfection, electroporation, liposome delivery, membrane fusion techniques, high velocity DNA-coated pellets, viral infection and protoplast fusion. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

The transgene or payload can be carried on any suitable vector, e.g., a plasmid, which is delivered to a host cell. The plasmids useful in this invention may be engineered such that they are suitable for replication and, optionally, integration in prokaryotic cells, mammalian cells, or both. These plasmids contain sequences permitting replication of the transgene in eukaryotes and/or prokaryotes and selection markers for these systems. Selectable markers or reporter genes may include sequences encoding geneticin, hygromicin or purimycin resistance, among others. The plasmids may also contain certain selectable reporters or marker genes that can be used to signal the presence of the vector in bacterial cells, such as ampicillin resistance. Other components of the plasmid may include an origin of replication and an amplicon, such as the amplicon system employing the Epstein Barr virus nuclear antigen. This amplicon system, or other similar amplicon components permit high copy episomal replication in the cells. Preferably, the molecule carrying the transgene or payload is transfected into the cell, where it may exist transiently. Alternatively, the transgene may be stably integrated into the genome of the host cell, either chromosomally or as an episome. In certain embodiments, the transgene may be present in multiple copies, optionally in head-to-head, head-to-tail, or tail-to-tail concatamers. Suitable transfection techniques are known and may readily be utilized to deliver the transgene to the host cell.

### Cells

The present disclosure provides a cell comprising a CRISPR-AAV polynucleotide and/or CRISPR-AAV genome.

Viral production of the invention disclosed herein describes processes and methods for producing CRISPR-AAV particles that contact a target cell to deliver a payload construct, e.g. a recombinant viral construct, which comprises a nucleotide encoding a payload molecule.

In one embodiment, the CRISPR-AAV particles of the invention may be produced in a viral replication cell that comprises an insect cell.

Growing conditions for insect cells in culture, and production of heterologous products in insect cells in culture are well-known in the art, see U.S. Pat. No. 6,204,059, the contents of which are herein incorporated by reference in their entirety.

Any insect cell which allows for replication of parvovirus and which can be maintained in culture can be used in accordance with the present invention. Cell lines may be used from Spodoptera frugiperda, including, but not limited to the Sf9 or Sf21 cell lines, drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of each of which are herein incorporated by reference in their entirety.

The viral replication cell may be selected from any biological organism, including prokaryotic (e.g., bacterial) cells, and eukaryotic cells, including, insect cells, yeast cells and mammalian cells. Viral replication cells may comprise mammalian cells such as HEK293, A549, WEH1, 3T3, 10T1/2, BHK, MDCK, COS 1, COS 7, BSC 1, BSC 40, BMT 10, VERO. W138, HeLa, 293, Saos, C2C12, L cells, HT1080, HepG2 and primary fibroblast, hepatocyte and myoblast cells derived from mammals. Viral replication cells of the invention comprise cells derived from mammalian species including, but not limited to, human, monkey, mouse, rat, rabbit, and hamster or cell type, including but not limited to fibroblast, hepatocyte, tumor cell, cell line transformed cell, etc.

### Small Scale Production of CRISPR-AAV Particles

Viral production of the invention disclosed herein describes processes and methods for producing CRISPR-AAV particles that contact a target cell to deliver a payload, e.g. a recombinant viral construct, which comprises a nucleotide encoding a payload.

In one embodiment, the CRISPR-AAV particles of the invention may be produced in a viral replication cell that comprises a mammalian cell.

Viral replication cells commonly used for production of recombinant AAV particles include, but is not limited to 293 cells, COS cells, HeLa cells, KB cells, and other mammalian cell lines as described in U.S. Pat. Nos. 6,156,303, 5,387,484, 5,741,683, 5,691,176, and 5,688,676; U.S. patent application 2002/0081721, and International Patent Applications WO 00/47757, WO 00/24916, and WO 96/17947, the contents of each of which are herein incorporated by reference in their entireties.

In one embodiment, CRISPR-AAV particles are produced in mammalian-cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In one embodiment the VP2 capsid may comprise a DNA binding domain for the cas9 promoter and/or a transactivating factor for cas9. The transactivating factor may be pre-engineered to induce cas9 expression.

In one embodiment, the expression of cas9 with a VP2 capsid comprising a DNA binding domain for the cas9 promoter and/or a transactivating factor for cas9 may be consistent over a period of time. The expression may be consistent for minutes (e.g., 1, 2, 3, 4, 5, 6, 7, 8, 9, 10, 15, 20, 25, 30, 35, 40, 45, 50, 55, or more than 55 minutes), hours (e.g., 1, 2, 3, 4, 5, 6, 7, 8, 9, 10, 11, 12, 13, 14, 15, 16, 17, 18, 19, 20, 21, 22, 23 or more than 24 hours), days (e.g., 1, 2, 3, 4, 5, 6, 7, 8, 9, 10, 11, 12, 13, 14, 15, 16, 17, 18, 19, 20 or more than 20 days), weeks (e.g., 1, 2, 3, 4, 5, 6, 7, 8, 9, 10 or more than 10 weeks), or months (e.g., 1, 2, 3, 4, 5, 6, 7, 8, 9, 10, 11, or more than 11 months). As another non-limiting example, expression of cas9 may be a burst of expression for a predetermined period of time (e.g., burst of expression for one hours, two hours, three hours, four hours, five hours, six hours or greater than six hours after administration).

In one embodiment, the promoter and/or transactiving factor may be located within the first 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. In another embodiment, the promoter and/or transactiving factor may be located within the last 1%, 2%, 3%, 4%, 5%, 6%, 7%, 8%, 9%, 10%, 15%, 20%, 25%, 30%, 35%, 40%, 45%, 46%, 47%, 48,% or 49% of the VP2 capsid. In another embodiment, the promoter and/or transactivating factor may be located in the middle of the VP2 capsid. In another embodiment, the promoter and/or transactiving factor may be located near the beginning of the VP2 capsid. In another embodiment, the promoter and/or transactiving factor may be located near the end of the VP2 capsid.

In another embodiment, CRISPR-AAV particles are produced in mammalian cells using a triple transfection method wherein a payload construct, parvoviral Rep, and parvoviral Cap are comprised within three different constructs. The triple transfection method of the three components of AAV particle production may be utilized to produce small lots of virus for assays including transduction efficiency, target tissue (tropism) evaluation, and stability.

### Baculovirus

Particle production of the invention disclosed herein describes processes and methods for producing CRISPR-AAV particles that contact a target cell to deliver a payload construct which comprises a nucleotide encoding a payload.

Briefly, the viral construct vector and the CRISPR-AAV payload construct vector of the invention are each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the CRISPR-AAV payload construct expression vector. The two baculoviruses may be used to infect a single viral replication cell population for production of CRISPR-AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and CRISPR-AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80 (4):1874-85, the contents of which are herein incorporated by reference in their entirety.

Production of CRISPR-AAV particles with baculovirus in an insect cell system may address known baculovirus genetic and physical instability. In one embodiment, the production system of the invention addresses baculovirus instability over multiple passages by utilizing a titerless infected-cells preservation and scale-up system. Small scale seed cultures of viral producing cells are transfected with viral expression constructs encoding the structural, non-structural, components of the viral particle. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the contents of which are herein incorporated by reference in their entirety.

A genetically stable baculovirus may be used to produce source of the one or more of the components for producing CRISPR-AAV particles in invertebrate cells. In one embodiment, defective baculovirus expression vectors may be maintained episomally in insect cells. In such an embodiment the bacmid vector is engineered with replication control elements, including but not limited to promoters, enhancers, and/or cell-cycle regulated replication elements.

In one embodiment, baculoviruses may be engineered with a (non-) selectable marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In one embodiment, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

### Large-Scale Production

In some embodiments, CRISPR-AAV particle production may be modified to increase the scale of production. Large scale viral production methods according to the present invention may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety. Methods of increasing viral particle production scale typically comprise increasing the number of viral replication cells. In some embodiments, viral replication cells comprise adherent cells. To increase the scale of viral particle production by adherent viral replication cells, larger cell culture surfaces are required. In some cases, large-scale production methods comprise the use of roller bottles to increase cell culture surfaces. Other cell culture substrates with increased surface areas are known in the art. Examples of additional adherent cell culture products with increased surface areas include, but are not limited to CELLSTACK®, CELLCUBE® (Corning Corp., Corning, N.Y.) and NUNC™ CELL FACTORY™ (Thermo Scientific, Waltham, Mass.) In some cases, large-scale adherent cell surfaces may comprise from about 1,000 cm² to about 100,000 cm². In some cases, large-scale adherent cell cultures may comprise from about 10⁷ to about 10⁹ cells, from about 10⁸ to about 10¹⁰ cells, from about 10⁹ to about 10¹² cells or at least 10¹² cells. In some cases, large-scale adherent cultures may produce from about 10⁹ to about 10¹², from about 10¹⁰ to about 10¹³, from about 10¹¹ to about 10¹⁴, from about 10¹² to about 10¹⁵ or at least 10¹⁵ viral particles.

In some embodiments, large-scale viral production methods of the present invention may comprise the use of suspension cell cultures. Suspension cell culture allows for significantly increased numbers of cells. Typically, the number of adherent cells that can be grown on about 10-50 cm² of surface area can be grown in about 1 cm³ volume in suspension.

Transfection of replication cells in large-scale culture formats may be carried out according to any methods known in the art. For large-scale adherent cell cultures, transfection methods may include, but are not limited to the use of inorganic compounds (e.g. calcium phosphate,) organic compounds [e.g. polyethylenimine (PEI)] or the use of non-chemical methods (e.g. electroporation.) With cells grown in suspension, transfection methods may include, but are not limited to the use of calcium phosphate and the use of PEI. In some cases, transfection of large scale suspension cultures may be carried out according to the section entitled “Transfection Procedure” described in Feng, L. et al., 2008. Biotechnol Appl. Biochem. 50:121-32, the contents of which are herein incorporated by reference in their entirety. According to such embodiments, PEI-DNA complexes may be formed for introduction of plasmids to be transfected. In some cases, cells being transfected with PEI-DNA complexes may be ‘shocked’ prior to transfection. This comprises lowering cell culture temperatures to 4° C. for a period of about 1 hour. In some cases, cell cultures may be shocked for a period of from about 10 minutes to about 5 hours. In some cases, cell cultures may be shocked at a temperature of from about 0° C. to about 20° C.

In some cases, transfections may include one or more vectors for expression of an RNA effector molecule to reduce expression of nucleic acids from one or more CRISPR-AAV payload construct. Such methods may enhance the production of viral particles by reducing cellular resources wasted on expressing payload constructs. In some cases, such methods may be carried according to those taught in US Publication No. US2014/0099666, the contents of which are herein incorporated by reference in their entirety.

### Bioreactors

In some embodiments, cell culture bioreactors may be used for large scale viral production. In some cases, bioreactors comprise stirred tank reactors. Such reactors generally comprise a vessel, typically cylindrical in shape, with a stirrer (e.g. impeller.) In some embodiments, such bioreactor vessels may be placed within a water jacket to control vessel temperature and/or to minimize effects from ambient temperature changes. Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In some cases, animal cell cultures may be maintained in bioreactors with rounded vessel bottoms.

In some cases, bioreactor vessels may be warmed through the use of a thermocirculator. Thermocirculators pump heated water around water jackets. In some cases, heated water may be pumped through pipes (e.g. coiled pipes) that are present within bioreactor vessels. In some cases, warm air may be circulated around bioreactors, including, but not limited to air space directly above culture medium. Additionally, pH and CO₂ levels may be maintained to optimize cell viability.

In some cases, bioreactors may comprise hollow-fiber reactors. Hollow-fiber bioreactors may support the culture of both anchorage dependent and anchorage independent cells. Further bioreactors may include, but are not limited to packed-bed or fixed-bed bioreactors. Such bioreactors may comprise vessels with glass beads for adherent cell attachment. Further packed-bed reactors may comprise ceramic beads.

In some cases, viral particles are produced through the use of a disposable bioreactor. In some embodiments, such bioreactors may include WAVE™ disposable bioreactors.

In some embodiments, CRISPR-AAV particle production in animal cell bioreactor cultures may be carried out according to the methods taught in U.S. Pat. Nos. 5,064,764, 6,194,191, 6,566,118, 8,137,948 or US Patent Application No. US2011/0229971, the contents of each of which are herein incorporated by reference in their entirety.

### Cell Lysis

Cells of the invention, including, but not limited to viral production cells, may be subjected to cell lysis according to any methods known in the art. Cell lysis may be carried out to obtain one or more agents (e.g. viral particles) present within any cells of the invention. In some embodiments, cell lysis may be carried out according to any of the methods listed in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety. Cell lysis methods may be chemical or mechanical. Chemical cell lysis typically comprises contacting one or more cells with one or more lysis agent. Mechanical lysis typically comprises subjecting one or more cells to one or more lysis condition and/or one or more lysis force.

In some embodiments, chemical lysis may be used to lyse cells. As used herein, the term “lysis agent” refers to any agent that may aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions may include one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may include one or more solubilizing agent. As used herein, the term “solubilizing agent” refers to a compound that enhances the solubility of one or more components of a solution and/or the solubility of one or more entities to which solutions are applied. In some cases, solubilizing agents enhance protein solubility. In some cases, solubilizing agents are selected based on their ability to enhance protein solubility while maintaining protein conformation and/or activity.

Exemplary lysis agents may include any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In some cases, lysis agents may be selected from lysis salts, amphoteric agents, cationic agents, ionic detergents and non-ionic detergents. Lysis salts may include, but are not limited to sodium chloride (NaCl) and potassium chloride (KCl.) Further lysis salts may include any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety. Concentrations of salts may be increased or decreased to obtain an effective concentration for rupture of cell membranes. Amphoteric agents, as referred to herein, are compounds capable of reacting as an acid or a base. Amphoteric agents may include, but are not limited to lysophosphatidylcholine, 3-((3-Cholamidopropyl) dimethylammonium)-1-propanesulfonate (CHAPS,) ZWITTERGENT® and the like. Cationic agents may include, but are not limited to cetyltrimethylammonium bromide (C (16) TAB) and Benzalkonium chloride. Lysis agents comprising detergents may include ionic detergents or non-ionic detergents. Detergents may function to break apart or dissolve cell structures including, but not limited to cell membranes, cell walls, lipids, carbohydrates, lipoproteins and glycoproteins. Exemplary ionic detergents include any of those taught in U.S. Pat. Nos. 7,625,570 and 6,593,123 or US Publication No. US2014/0087361, the contents of each of which are herein incorporated by reference in their entirety. Some ionic detergents may include, but are not limited to sodium dodecyl sulfate (SDS), cholate and deoxycholate. In some cases, ionic detergents may be included in lysis solutions as a solubilizing agent. Non-ionic detergents may include, but are not limited to octylglucoside, digitonin, lubrol, C12E8, TWEEN®-20, TWEEN®-80, Triton X-100 and Noniodet P-40. Non-ionic detergents are typically weaker lysis agents, but may be included as solubilizing agents for solubilizing cellular and/or viral proteins. Further lysis agents may include enzymes and urea. In some cases, one or more lysis agents may be combined in a lysis solution in order to enhance one or more of cell lysis and protein solubility. In some cases, enzyme inhibitors may be included in lysis solutions in order to prevent proteolysis that may be triggered by cell membrane disruption.

In some embodiments, mechanical cell lysis is carried out. Mechanical cell lysis methods may include the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In some cases, lysis conditions comprise increased or decreased temperatures. According to some embodiments, lysis conditions comprise changes in temperature to promote cellular disruption. Cell lysis carried out according to such embodiments may include freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In some cases, one or more cyroprotectants are included in cell solutions undergoing freeze-thaw lysis. As used herein, the term “cryoprotectant” refers to an agent used to protect one or more substance from damage due to freezing. Cryoprotectants of the invention may include any of those taught in US Publication No. US2013/0323302 or U.S. Pat. Nos. 6,503,888, 6,180,613, 7,888,096, 7,091,030, the contents of each of which are herein incorporated by reference in their entirety. In some cases, cryoprotectants may include, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In some embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may include, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may include high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g. viral particles.) Other mechanical lysis methods may include physical disruption of cells by scraping.

Cell lysis methods may be selected based on the cell culture format of cells to be lysed. For example, with adherent cell cultures, some chemical and mechanical lysis methods may be used. Such mechanical lysis methods may include freeze-thaw lysis or scraping. In another example, chemical lysis of adherent cell cultures may be carried out through incubation with lysis solutions comprising surfactant, such as Triton-X-100. In some cases, cell lysates generated from adherent cell cultures may be treated with one more nuclease to lower the viscosity of the lysates caused by liberated DNA.

In one embodiment, a method for harvesting CRISPR-AAV particles without lysis may be used for efficient and scalable CRISPR-AAV particle production. In a non-limiting example, CRISPR-AAV particles may be produced by culturing a CRISPR-AAV particle lacking a heparin binding site, thereby allowing the CRISPR-AAV particle to pass into the supernatant, in a cell culture, collecting supernatant from the culture; and isolating the CRISPR-AAV particle from the supernatant, as described in US Patent Application 20090275107, the contents of which is incorporated herein by reference in its entirety.

### Clarification

Cell lysates comprising viral particles may be subjected to clarification. Clarification refers to initial steps taken in purification of viral particles from cell lysates. Clarification serves to prepare lysates for further purification by removing larger, insoluble debris. Clarification steps may include, but are not limited to centrifugation and filtration. During clarification, centrifugation may be carried out at low speeds to remove larger debris, only. Similarly, filtration may be carried out using filters with larger pore sizes so that only larger debris is removed. In some cases, tangential flow filtration may be used during clarification. Objectives of viral clarification include high throughput processing of cell lysates and to optimize ultimate viral recovery. Advantages of including a clarification step include scalability for processing of larger volumes of lysate. In some embodiments, clarification may be carried out according to any of the methods presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

Methods of cell lysate clarification by filtration are well understood in the art and may be carried out according to a variety of available methods including, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may include, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In one embodiment, clarification may comprise filtration through a filter with 2.0 μM pore size to remove large debris, followed by passage through a filter with 0.45 μM pore size to remove intact cells.

Filter materials may be composed of a variety of materials. Such materials may include, but are not limited to polymeric materials and metal materials (e.g. sintered metal and pored aluminum.) Exemplary materials may include, but are not limited to nylon, cellulose materials (e.g. cellulose acetate), polyvinylidene fluoride (PVDF), polyethersulfone, polyamide, polysulfone, polypropylene, and polyethylene terephthalate. In some cases, filters useful for clarification of cell lysates may include, but are not limited to ULTIPLEAT PROFILE™ filters (Pall Corporation, Port Washington, N.Y.), SUPOR™ membrane filters (Pall Corporation, Port Washington, N.Y.)

In some cases, flow filtration may be carried out to increase filtration speed and/or effectiveness. In some cases, flow filtration may comprise vacuum filtration. According to such methods, a vacuum is created on the side of the filter opposite that of cell lysate to be filtered. In some cases, cell lysates may be passed through filters by centrifugal forces. In some cases, a pump is used to force cell lysate through clarification filters. Flow rate of cell lysate through one or more filters may be modulated by adjusting one of channel size and/or fluid pressure.

According to some embodiments, cell lysates may be clarified by centrifugation. Centrifugation may be used to pellet insoluble particles in the lysate. During clarification, centrifugation strength [expressed in terms of gravitational units (g), which represents multiples of standard gravitational force] may be lower than in subsequent purification steps. In some cases, centrifugation may be carried out on cell lysates at from about 200 g to about 800 g, from about 500 g to about 1500 g, from about 1000 g to about 5000 g, from about 1200 g to about 10000 g or from about 8000 g to about 15000 g. In some embodiments, cell lysate centrifugation is carried out at 8000 g for 15 minutes. In some cases, density gradient centrifugation may be carried out in order to partition particulates in the cell lysate by sedimentation rate. Gradients used according to methods of the present invention may include, but are not limited to cesium chloride gradients and iodixanol step gradients.

### Purification: Chromatography

In some cases, CRISPR-AAV particles may be purified from clarified cell lysates by one or more methods of chromatography. Chromatography refers to any number of methods known in the art for separating out one or more elements from a mixture. Such methods may include, but are not limited to ion exchange chromatography (e.g. cation exchange chromatography and anion exchange chromatography,) immunoaffinity chromatography and size-exclusion chromatography. In some embodiments, methods of viral chromatography may include any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

In some embodiments, ion exchange chromatography may be used to isolate viral particles. Ion exchange chromatography is used to bind viral particles based on charge-charge interactions between capsid proteins and charged sites present on a stationary phase, typically a column through which viral preparations (e.g. clarified lysates) are passed. After application of viral preparations, bound viral particles may then be eluted by applying an elution solution to disrupt the charge-charge interactions. Elution solutions may be optimized by adjusting salt concentration and/or pH to enhance recovery of bound viral particles. Depending on the charge of viral capsids being isolated, cation or anion exchange chromatography methods may be selected. Methods of ion exchange chromatography may include, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In some embodiments, immunoaffinity chromatography may be used. Immunoaffinity chromatography is a form of chromatography that utilizes one or more immune compounds (e.g. antibodies or antibody-related structures) to retain viral particles. Immune compounds may bind specifically to one or more structures on viral particle surfaces, including, but not limited to one or more viral coat protein. In some cases, immune compounds may be specific for a particular viral variant. In some cases, immune compounds may bind to multiple viral variants. In some embodiments, immune compounds may include recombinant single-chain antibodies. Such recombinant single chain antibodies may include those described in Smith, R. H. et al., 2009. Mol. Ther. 17(11):1888-96, the contents of which are herein incorporated by reference in their entirety. Such immune compounds are capable of binding to several AAV capsid variants, including, but not limited to AAV1, AAV2, AAV6 and AAV8.

In some embodiments, size-exclusion chromatography (SEC) may be used. SEC may comprise the use of a gel to separate particles according to size. In viral particle purification, SEC filtration is sometimes referred to as “polishing.” In some cases, SEC may be carried out to generate a final product that is near-homogenous. Such final products may in some cases be used in pre-clinical studies and/or clinical studies (Kotin, R. M. 2011. Human Molecular Genetics. 20(1):R2-R6, the contents of which are herein incorporated by reference in their entirety.) In some cases, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In one embodiment, the compositions comprising at least one CRISPR-AAV particle may be isolated or purified using the methods described in U.S. Pat. No. 6,146,874, the contents of which are herein incorporated by reference in its entirety.

In one embodiment, the compositions comprising at least one CRISPR-AAV particle may be isolated or purified using the methods described in U.S. Pat. No. 6,660,514, the contents of which are herein incorporated by reference in its entirety.

In one embodiment, the compositions comprising at least one CRISPR-AAV particle may be isolated or purified using the methods described in U.S. Pat. No. 8,283,151, the contents of which are herein incorporated by reference in its entirety.

In one embodiment, the compositions comprising at least one CRISPR-AAV particle may be isolated or purified using the methods described in U.S. Pat. No. 8,524,446, the contents of which are herein incorporated by reference in its entirety.

### Gene Silencing: Knockdown Approach

When designed to inhibit or silence a gene, the CRISPR-AAV particles of the present invention may also comprise a viral genome that encodes a polynucleotide payload which may be processed to produce an siRNA, miRNA or other double stranded (ds) or single stranded (ss) gene modulatory motif.

Accordingly, the siRNA duplexes or dsRNA can be used to substantially inhibit gene expression in a cell, in particular cells of the CNS. In some aspects, the inhibition of gene expression refers to an inhibition by at least about 20%, preferably by at least about 30%, 40%, 50%, 60%, 70%, 80%, 85%, 90%, 95% and 100%. Accordingly, the protein product of the targeted gene may be inhibited by at least about 20%, preferably by at least about 30%, 40%, 50%, 60%, 70%, 80%, 85%, 90%, 95% and 100%. The gene can be either a wild type gene or a mutated gene with at least one mutation. Accordingly, the protein is either wild type protein or a mutated polypeptide with at least one mutation.

In some embodiments, the present invention provides methods for treating, or ameliorating a disease or condition associated with abnormal gene and/or protein in a subject in need of treatment, the method comprising administering to the subject an effective amount of at least one CRISPR-AAV particle comprising a viral genome encoding an siRNA duplex targeting the abnormal gene and/or protein, delivering duplex into targeted cells, inhibiting the gene expression and protein production, and ameliorating symptoms of the disease or condition in the subject.

### Gene Replacement or Activation

When designed to increase the expression of a gene or replace a gene, CRISPR-AAV particles may comprise a viral genome encoding a payload which encodes a normal gene to replace a mutated, defective or nonfunctional copy of that gene in the recipient.

### Treatment and Pharmaceutical Compositions

The present disclosure additionally provides a method for treating a disease, disorder and/or condition in a mammalian subject, including a human subject, comprising administering to the subject any of the CRISPR-AAV polynucleotides or CRISPR-AAV genomes described herein (i.e., viral genomes or “VG”) or administering to the subject a particle comprising the CRISPR-AAV polynucleotide or CRISPR-AAV genome, or administering to the subject any of the described compositions, including pharmaceutical compositions. In one embodiment, the disease, disorder and/or condition is a neurological disease, disorder and/or condition. In one embodiment, the neurological disease, disorder and/or condition is Parkinson's disease. In another embodiment, the neurological disease, disorder and/or condition is Friedreich's Ataxia. In another embodiment, the neurological disease, disorder and/or condition is Amyotrophic lateral sclerosis (ALS). In another embodiment, the neurological disease, disorder and/or condition is Huntington's disease. In another embodiment, the neurological disease, disorder or condition is spinal muscular atrophy (SMA). In another embodiment, the disease, disorder and/or condition is a muscular or cardiac disease, disorder and/or condition. In another embodiment, the disease, disorder and/or condition is an immune system disease, disorder and/or condition.

As used herein the term “composition” comprises a CRISPR-AAV polynucleotide or CRISPR-AAV genome or CRISPR-AAV particle and at least one excipient.

As used herein the term “pharmaceutical composition” comprises a CRISPR-AAV polynucleotide or CRISPR-AAV genome or CRISPR-AAV particle and one or more pharmaceutically acceptable excipients.

Although the descriptions of pharmaceutical compositions, e.g., those CRISPR-AAV particles comprising a payload to be delivered, provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, human patients or subjects. For the purposes of the present disclosure, the phrase “active ingredient” generally refers either to the viral particle carrying the payload or to the payload delivered by the viral particle as described herein.

Formulations of the CRISPR-AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered.

### Formulation

The CRISPR-AAV particles of the invention can be formulated using one or more excipients to: (1) increase stability; (2) increase cell transfection or transduction; (3) permit the sustained or delayed release; (4) alter the biodistribution (e.g., target the viral particle to specific tissues or cell types); (5) increase the translation of encoded protein in vivo; (6) alter the release profile of encoded protein in vivo; (7) allow for regulatable expression of the payload and/or (8) allow for short-lived, transient, or burst-like expression of a CRISPR regulatable element (e.g., a cas9 element).

Formulations of the present invention can include, without limitation, saline, liposomes, lipid nanoparticles, polymers, lipoplexes, core-shell nanoparticles, peptides, proteins, cells transfected with viral vectors (e.g., for transplantation into a subject), nanoparticle mimics and combinations thereof.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g. CRISPR-AAV), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the CRISPR-AAV formulations described herein may contain at least one payload. As a non-limiting example, the formulations may contain 1, 2, 3, 4 or 5 payloads. In one embodiment the formulation may contain a payload encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the formulation contains at least three CRISPR-AAV payload encoding proteins.

The formulations of the invention can include one or more excipients, each in an amount that together increases the stability of the CRISPR-AAV particle, increases cell transfection or transduction by the viral particle, increases the expression of viral polynucleotide encoded protein, and/or alters the release profile of CRISPR-AAV polynucleotide encoded proteins. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Inactive Ingredients

In some embodiments, CRISPR-AAV formulations may comprise at least one excipient which is an inactive ingredient. As used herein, the term “inactive ingredient” refers to one or more inactive agents included in formulations. In some embodiments, all, none or some of the inactive ingredients which may be used in the formulations of the present invention may be approved by the US Food and Drug Administration (FDA).

Formulations of CRISPR-AAV particles disclosed herein may include cations or anions. In one embodiment, the formulations include metal cations such as, but not limited to, Zn2+, Ca2+, Cu2+, Mg+ and combinations thereof.

### Administration

The CRISPR-AAV particles of the present invention may be administered by any route which results in a therapeutically effective outcome. These include, but are not limited to enteral (into the intestine), gastroenteral, epidural (into the dura mater), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them cross the blood-brain barrier, vascular barrier, or other epithelial barrier. In one embodiment, a formulation for a route of administration may include at least one inactive ingredient.

### Dosing

The present invention provides methods of administering CRISPR-AAV particles and their payload or complexes in accordance with the invention to a subject in need thereof. CRISPR-AAV particle pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in unit dosage form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific payload employed; the duration of the treatment; drugs used in combination or coincidental with the specific payload employed; and like factors well known in the medical arts.

In certain embodiments, CRISPR-AAV particle pharmaceutical compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. It will be understood that the above dosing concentrations may be converted to vg or viral genomes per kg or into total viral genomes administered by one of skill in the art.

The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hour period. It may be administered as a single unit dose. In one embodiment, the viral particles of the present invention are administered to a subject in split doses. The viral particles may be formulated in buffer only or in a formulation described herein.

A pharmaceutical composition described herein can be formulated into a dosage form described herein, such as a topical, intranasal, pulmonary, intratracheal, or injectable (e.g., intravenous, intraocular, intravitreal, intramuscular, intracardiac, intraperitoneal, and/or subcutaneous).

### Combinations

The CRISPR-AAV particles may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

### Delivery to Cells

The present disclosure provides a method of delivering to a cell or tissue any of the above-described CRISPR-AAV polynucleotides or CRISPR-AAV genomes, comprising contacting the cell or tissue with the CRISPR-AAV polynucleotide or CRISPR-AAV genomes or contacting the cell or tissue with a particle comprising the CRISPR-AAV polynucleotide or CRISPR-AAV genome, or contacting the cell or tissue with any of the described compositions, including pharmaceutical compositions. The method of delivering the CRISPR-AAV polynucleotide or CRISPR-AAV genome to a cell or tissue can be accomplished in vitro, ex vivo, or in vivo.

### Delivery to Subjects

The present disclosure additionally provides a method of delivering to a subject, including a mammalian subject, any of the above-described CRISPR-AAV polynucleotides or CRISPR-AAV genomes comprising administering to the subject the CRISPR-AAV polynucleotide or CRISPR-AAV genome, or administering to the subject a particle comprising the CRISPR-AAV polynucleotide or CRISPR-AAV genome, or administering to the subject any of the described compositions, including pharmaceutical compositions.

### Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Adeno-associated virus: The term “adeno-associated virus” or “AAV” as used herein refers to members of the dependovirus genus comprising any particle, sequence, gene, protein, or component derived therefrom. The term “AAV particle” as used herein comprises a capsid and a polynucleotide. The AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted. Where CRISPR regulatable elements are present, the AAV is a CRISPR-AAV. The term “CRISPR-AAV particle” as used herein comprises a capsid, a polynucleotide, and one or more CRISPR regulatable elements, which may be encoded by the polynucleotide or extant to the polynucleotide.

Activity: As used herein, the term “activity” refers to the condition in which things are happening or being done. Compositions of the invention may have activity and this activity may involve one or more biological events.

Administered in combination: As used herein, the term “administered in combination” or “combined administration” refers to simultaneous exposure to two or more agents (e.g., CRISPR-AAV) administered at the same time or within an interval such that the subject is at some point in time simultaneously exposed to both and/or such that there may be an overlap in the effect of each agent on the patient. In some embodiments, at least one dose of one or more agents is administered within about 24 hours, 12 hours, 6 hours, 3 hours, 1 hour, 30 minutes, 15 minutes, 10 minutes, 5 minutes, or 1 minute of at least one dose of one or more other agents. In some embodiments, administration occurs in overlapping dosage regimens. As used herein, the term “dosage regimen” refers to a plurality of doses spaced apart in time. Such doses may occur at regular intervals or may include one or more hiatus in administration. In some embodiments, the administration of individual doses of one or more compounds and/or compositions of the present invention, as described herein, are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biomolecule: As used herein, the term “biomolecule” is any natural molecule which is amino acid-based, nucleic acid-based, carbohydrate-based or lipid-based, and the like.

Biologically active: As used herein, the phrase “biologically active” refers to a characteristic of any substance (e.g., a CRISPR-AAV) that has activity in or on a biological system and/or organism. For instance, a substance that, when administered to an organism, has a biological effect on that organism, is considered to be biologically active. In particular embodiments, a compounds and/or compositions of the present invention may be considered biologically active if even a portion of is biologically active or mimics an activity considered to biologically relevant.

Biological system: As used herein, the term “biological system” refers to a group of organs, tissues, cells, intracellular components, proteins, nucleic acids, molecules (including, but not limited to biomolecules) that function together to perform a certain biological task within cellular membranes, cellular compartments, cells, tissues, organs, organ systems, multicellular organisms, or any biological entity. In some embodiments, biological systems are cell signaling pathways comprising intracellular and/or extracellular cell signaling biomolecules. In some embodiments, biological systems comprise growth factor signaling events within the extracellular/cellular matrix and/or cellular niches.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: As used herein, the term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art appreciate that some compounds exist in different such forms, show different properties and/or activities (including, but not limited to biological activities). In such cases it is within the ordinary skill of those in the art to select or avoid particular forms of the compound for use in accordance with the present invention. For example, compounds that contain asymmetrically substituted carbon atoms can be isolated in optically active or racemic forms. Methods on how to prepare optically active forms from optically active starting materials are known in the art, such as by resolution of racemic mixtures or by stereoselective synthesis.

Conserved: As used herein, the term “conserved” refers to nucleotides or amino acid residues of polynucleotide or polypeptide sequences, respectively, that are those that occur unaltered in the same position of two or more sequences being compared. Nucleotides or amino acids that are relatively conserved are those that are conserved among more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

In one embodiment, conserved sequences are not contiguous. Those skilled in the art are able to appreciate how to achieve alignment when gaps in contiguous alignment are present between sequences, and to align corresponding residues not withstanding insertions or deletions present.

Delivery: As used herein, “delivery” refers to the act or manner of delivering parvovirus, e.g. an AAV and/or CRISPR-AAV compound, substance, entity, moiety, cargo or payload to a target. Such target may be a cell, tissue, organ, organism, or system (whether biological or production).

Delivery Agent: As used herein, “delivery agent” refers to any agent which facilitates, at least in part, the delivery of one or more substances (including, but not limited to a compounds and/or compositions of the present invention, e.g., viral particles or expression vectors) to targeted cells.

Destabilized: As used herein, the term “destable,” “destabilize,” or “destabilizing region” means a region or molecule that is less stable than a starting, reference, wild-type or native form of the same region or molecule.

Detectable label: As used herein, “detectable label” refers to one or more markers, signals, or moieties which are attached, incorporated or associated with another entity, which markers, signals or moieties are readily detected by methods known in the art including radiography, fluorescence, chemiluminescence, enzymatic activity, absorbance, immunological detection and the like. Detectable labels may include radioisotopes, fluorophores, chromophores, enzymes, dyes, metal ions, ligands, biotin, avidin, streptavidin and haptens, quantum dots, polyhistidine tags, myc tags, flag tags, human influenza hemagglutinin (HA) tags and the like. Detectable labels may be located at any position in the entity with which they are attached, incorporated or associated. For example, when attached, incorporated in or associated with a peptide or protein, they may be within the amino acids, the peptides, or proteins, or located at the N- or C-termini.

Effective amount: As used herein, the term “effective amount” of an agent is that amount sufficient to effect beneficial or desired results, for example, upon single or multiple dose administration to a subject cell, in curing, alleviating, relieving or improving one or more symptoms of a disorder and, as such, an “effective amount” depends upon the context in which it is being applied. For example, in the context of administering an agent that treats ALS, an effective amount of an agent is, for example, an amount sufficient to achieve treatment, as defined herein, of ALS, as compared to the response obtained without administration of the agent.

Engineered: As used herein, embodiments of the invention are “engineered” when they are designed to have a feature or property, whether structural or chemical, that varies from a starting point, wild-type or native molecule. Thus, engineered agents or entities are those whose design and/or production include an act of the hand of man.

Epitope: As used herein, an “epitope” refers to a surface or region on a molecule that is capable of interacting with a biomolecule. For example a protein may contain one or more amino acids, e.g., an epitope, which interacts with an antibody, e.g., a biomolecule. In some embodiments, when referring to a protein or protein module, an epitope may comprise a linear stretch of amino acids or a three dimensional structure formed by folded amino acid chains.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Feature: As used herein, a “feature” refers to a characteristic, a property, or a distinctive element.

Formulation: As used herein, a “formulation” includes at least a compound and/or composition of the present invention (e.g., a vector, CRISPR-AAV particle, etc.) and a delivery agent.

Fragment: A “fragment,” as used herein, refers to a contiguous portion of a whole. For example, fragments of proteins may comprise polypeptides obtained by digesting full-length protein isolated from cultured cells. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody subjected to enzymatic digestion or synthesized as such.

Functional: As used herein, a “functional” biological molecule is a biological entity with a structure and in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is typically determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids. In many embodiments, homologous protein may show a large overall degree of homology and a high degree of homology over at least one short stretch of at least 3, 4, 5, 6, 7, 8, 9, 10, 11, 12, 13, 14, 15, 16, 17, 18, 19, 20, 25, 30, 35, 40, 45, 50 or more amino acids. In many embodiments, homologous proteins share one or more characteristic sequence elements. As used herein, the term “characteristic sequence element” refers to a motif present in related proteins. In some embodiments, the presence of such motifs correlates with a particular activity (such as biological activity).

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined, for example using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product may be RNA transcribed from the gene (e.g. mRNA) or a polypeptide translated from mRNA transcribed from the gene. Typically a reduction in the level of mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” is synonymous with “separated”, but carries with it the inference separation was carried out by the hand of man. In one embodiment, an isolated substance or entity is one that has been separated from at least some of the components with which it was previously associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art. In some embodiments, isolation of a substance or entity includes disruption of chemical associations and/or bonds. In some embodiments, isolation includes only the separation from components with which the isolated substance or entity was previously combined and does not include such disruption.

Modified: As used herein, the term “modified” refers to a changed state or structure of a molecule or entity as compared with a parent or reference molecule or entity. Molecules may be modified in many ways including chemically, structurally, and functionally. In some embodiments, compounds and/or compositions of the present invention are modified by the introduction of non-natural amino acids, or non-natural nucleotides.

Mutation: As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids). In embodiments wherein mutations comprise the addition and/or substitution of amino acids and/or nucleotides, such additions and/or substitutions may comprise 1 or more amino acid and/or nucleotide residues and may include modified amino acids and/or nucleotides.

Naturally occurring: As used herein, “naturally occurring” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic acid: As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA.

Off-target: As used herein, “off target” refers to any unintended effect on any one or more target, gene and/or cellular transcript.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid.

Patient: As used herein, “patient” refers to a subject who may seek or be in need of treatment, requires treatment, is receiving treatment, will receive treatment, or a subject who is under care by a trained (e.g., licensed) professional for a particular disease or condition.

Payload: As used herein, “payload” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. Where CRISPR regulatable elements are encoded, the payload is a “CRISPR-AAV payload.”

Payload construct: As used herein, “payload construct” is one or more polynucleotide regions encoding or comprising a payload that is flanked on one or both sides by an inverted terminal repeat (ITR) sequence. The payload construct is a template that is replicated in a viral production cell to produce a viral genome. Where CRISPR regulatable elements are encoded, the payload construct is a “CRISPR-AAV payload construct.”

Payload construct vector: As used herein, “payload construct vector” is a vector encoding or comprising a payload construct, and regulatory regions for replication and expression in bacterial cells. Where CRISPR regulatable elements are encoded, the payload construct vector is a “CRISPR-AAV payload construct vector.”

Payload construct expression vector: As used herein, a “payload construct expression vector” is a vector encoding or comprising a payload construct and which further comprises one or more polynucleotide regions encoding or comprising components for viral expression in a viral replication cell. Where CRISPR regulatable elements are encoded, the payload construct expression vector is a “CRISPR-AAV payload construct expression vector.”

Peptide: As used herein, the term “peptide” refers to a chain of amino acids that is less than or equal to about 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids. In some embodiments a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety. Pharmaceutically acceptable solvate: The term “pharmaceutically acceptable solvate,” as used herein, refers to a crystalline form of a compound wherein molecules of a suitable solvent are incorporated in the crystal lattice. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.” In some embodiments, the solvent incorporated into a solvate is of a type or at a level that is physiologically tolerable to an organism to which the solvate is administered (e.g., in a unit dosage form of a pharmaceutical composition).

Pharmacokinetic: As used herein, “pharmacokinetic” refers to any one or more properties of a molecule or compound as it relates to the determination of the fate of substances administered to living organisms. Pharmacokinetics are divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand, replicate or increase or cause to grow, expand, replicate or increase. “Proliferative” means having the ability to proliferate. “Anti-proliferative” means having properties counter to or in opposition to proliferative properties.

Protein of interest: As used herein, the terms “proteins of interest” or “desired proteins” include those provided herein and fragments, mutants, variants, and alterations thereof.

Purified: As used herein, the term “purify” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interference: As used herein, the term “RNA interference” or “RNAi” refers to a sequence specific regulatory mechanism mediated by RNA molecules which results in the inhibition or interference or “silencing” of the expression of a corresponding protein-coding gene.

Sample: As used herein, the term “sample” refers to an aliquot or portion taken from a source and/or provided for analysis or processing. In some embodiments, a sample is from a biological source such as a tissue, cell or component part (e.g. a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or comprise a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In some embodiments, a sample is or comprises a medium, such as a nutrient broth or gel, which may contain cellular components, such as proteins or nucleic acid molecule. In some embodiments, a “primary” sample is an aliquot of the source. In some embodiments, a primary sample is subjected to one or more processing (e.g., separation, purification, etc.) steps to prepare a sample for analysis or other use.

Self-complementary viral particle: As used herein, a “self-complementary viral particle” is a particle comprised of at least two components, a protein capsid and a polynucleotide sequence encoding a self-complementary genome enclosed within the capsid.

Sense strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the siRNA strand.

Signal Sequences: As used herein, the phrase “signal sequences” refers to a sequence which can direct the transport or localization.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Small/short interfering RNA: As used herein, the term “small/short interfering RNA” or “siRNA” refers to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Split dose: As used herein, a “split dose” is the division of single unit dose or total daily dose into two or more doses.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a reference compound or entity.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term typically means within about 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Synthetic: The term “synthetic” means produced, prepared, and/or manufactured by the hand of man. Synthesis of polynucleotides or polypeptides or other molecules of the present invention may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in a 24 hour period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Vector: As used herein, a “vector” is any molecule or moiety which transports, transduces or otherwise acts as a carrier of a heterologous molecule.

Vectors of the present invention may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral construct vector: As used herein, a “viral construct vector” is a vector which comprises one or more polynucleotide regions encoding or comprising Rep and or Cap protein.

Viral construct expression vector: As used herein, a “viral construct expression vector” is a vector which comprises one or more polynucleotide regions encoding or comprising Rep and or Cap that further comprises one or more polynucleotide regions encoding or comprising components for viral expression in a viral replication cell.

Viral genome: As used herein, a “viral genome” is a polynucleotide encoding at least one inverted terminal repeat (ITR), at least one regulatory sequence, and at least one payload. The viral genome is derived by replication of a payload construct from the payload construct expression vector. A viral genome encodes at least one copy of the payload construct. Where CRISPR regulatable elements are encoded, a viral genome encodes at least one copy of the CRISPR-AAV payload construct.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

### EXAMPLES

### Example 1. Gene Expression

The level of transgene expression by CRISPR-AAV particles produced and purified by the methods described herein is determined by real-time quantitative polymerase chain reaction (qPCR). A culture of 293 cells engineered to produce helper components required for AAV production is infected by CRISPR-AAV particles produced as described herein.

The target 293 cells are harvested at a series of time points, lysed and the mRNA is purified. The level of transgene expressed is determined by reverse transcription (qPCR) on a thermal cycler equipped with an excitation source filters, and detector for quantification of the reaction such as, but not limited to, the 7500 FAST Real-Time PCR system (Applied Biosystems, Foster City Calif.).

CRISPR-AAV particles produced and purified by the methods described herein is treated with proteinase K, serially diluted, and PCR-amplified using a fluor such as, but not limited to, SYBR green (Applied Biosystems, Foster City, Calif.) with primers specific to the transgene sequence. A reference transgene oligonucleotide is used as a copy number standard. The cycling conditions are: 95° C. for 3 min, followed by 35 cycles of 95° C. for 30 sec, 60° C. for 30 sec, and 72° C. for 30 sec.

### Example 2. Recombinant CRISPR-AAV Production in Invertebrate Cells

The AAV viral construct vector encodes the three structural cap proteins, VP1, VP2, and VP3, in a single open reading frame regulated by utilization of both alternative splice acceptor and non-canonical translational initiation codon(s). In-frame and out-of-frame ATG triplets preventing translation initiation at a position between the VP1 and VP2 start codons are eliminated. Both Rep78 and Rep52 are translated from a single transcript: Rep78 translation initiates at a non-AUG codon and Rep52 translation initiates at the first AUG in the transcript.

The nucleotides that encode the structural VP1, VP2, and VP3 capsid proteins and non-structural Rep78 and Rep52 proteins are contained on one viral expression construct under control of the baculovirus major late promoter.

The payload construct vector encodes two ITR sequences flanking a transgene polynucleotide encoding a polypeptide or modulatory nucleic acid and/or one or more CRISPR regulatable elements. The ITR sequences allow for replication of a polynucleotide encoding the transgene and ITR sequences alone that will be packaged within the capsid of the viral vector. The replicated polynucleotide encodes ITR sequences on the 5′ and 3′ ends of the molecule.

The payload construct vector and viral construct vector each comprise a Tn7 transposon element that transposes the ITR-payload sequences or the Rep and Cap sequences respectively to a bacmid that comprises the attTn7 attachment site. Competent bacterial DH10 cells are transfected with either the payload construct vector or viral construct vector. The resultant viral construct expression vector and payload construct expression vector produced in the competent cell are then purified by detergent lysis and purification on DNA columns.

Separate seed cultures of Sf9 cells in serum free suspension culture are transfected with the viral construct expression vector or payload construct expression vector. The cultures are maintained for 48 hours while baculovirus is produced and released into the medium. The baculovirus released into the media continue to infect Sf9 cells in an exponential manner until all of the Sf9 cells in the culture are infected at least once. The baculoviral infected insect cells (BIIC) and media of the seed culture is harvested and divided into aliquots before being frozen in liquid nitrogen.

A naïve population of un-transfected Sf9 cells is expanded in serum free suspension cell culture conditions. Once the culture growth has reached peak log phase in 1 L of media as measured by optical density the culture is added to a large volume 20 L bioreactor. The bioreactor culture is co-inoculated with a frozen viral construct expression vector and payload construct expression vector BIIC aliquot. The conditions of the Sf9 cell suspension culture is monitored by instruments that measure and/or control external variables that support the growth and activity of viral replication cells such as mass, temperature, CO₂, O₂, pH, and/or optical density (OD). The Sf9 culture is maintained at optimal conditions until cell population growth has reached peak log phase and before cell growth has plateaued, as measured by optical density.

In each viral replication cell that has been infected with both baculoviruses the payload flanked on one end with an ITR sequence is replicated pathway producing a CRISPR-AAV genome and packaged in a capsid assembled from the proteins VP1, VP2, and VP3.

The viral replication cells are lysed using the Microfluidizer™ (Microfluidics International Corp., Newton, Mass.), high shear force fluid processor. The resultant cell lysate is clarified by low speed centrifugation followed by tangential flow filtration. The resultant clarified lysate is filtered by a size exclusion column to remove any remaining baculoviral particles from solution. The final steps utilize ultracentrifugation and sterile filtration to produce viral particles suitable for use as described herein.

The titer of CRISPR-AAV particles produced and purified by the methods described herein is determined by real-time quantitative polymerase chain reaction (qPCR) on a thermal cycler equipped with an excitation source filters, and detector for quantification of the reaction such as, but not limited to, the 7500 FAST Real-Time PCR system (Applied Biosystems, Foster City Calif.). CRISPR-AAV particles produced and purified by the methods described herein is treated with proteinase K, serially diluted, and PCR-amplified using a fluor such as, but not limited to, SYBR green (Applied Biosystems, Foster City, Calif.) with primers specific to the AAV genome ITR sequences. Linearized AAV vector genome is used as a copy number standard. The cycling conditions are: 95° C. for 3 min, followed by 35 cycles of 95° C. for 30 sec, 60° C. for 30 sec, and 72° C. for 30 sec.

### Example 3. Incorporation of Destabilizing Sequences or Domains

To minimize long-term dsDNA cleavage, CRISPR-AAV constructs with destabilizing domains and destabilizing RNA sequences are prepared, produced and tested.

### A. Destabilizing Domains—Cas9 Fusion Protein

### Overview

While not wishing to be bound by theory, destabilizing domains are known to confer instability and decrease transgene expression. The presence of the destabilizing domain can trigger the cell's proteasomal degradation systems, which then can lead to cas9 destruction. Destabilizing domains may comprise peptide sequences which are rich in a particular subset of amino acids which are thought to signal degradation, such as, but not limited to, proline, glutamic acid, serine and threonine (known as PEST sequences).

In some embodiments, destabilizing domains, which can be used in the cas9-fusion protein encoded by the CRISPR-AAV payload construct expression vector include, but are not limited to, destabilizing domains from FK506 Binding Protein (FKBP), E. coli dihyrofolate reductase (DHFR), mouse ornithine decarboxylase (MODC), and estrogen receptors (ER). As a non-limiting example, the destabilizing domain may be from an estrogen receptor.

### AAV Construct Preparation and Analysis

In order to study methods to minimize unwanted long-term cleavage of dsDNA, a CRISPR-AAV payload construct expression vector of Example 2 with a cas9-fusion protein (e.g., a cas9 endonuclease fused to a destabilizing domain) is prepared, produced and tested by methods known in the art and described herein.

The decreased protein half-life of cas9 endonuclease with a destabilizing domain is tested by infecting HEK293 cells with the CRISPR-AAV payload construct expression vectors comprising cas9 with or without destabilizing domain. Cells are lysed over a predetermined time course (e.g., 24 hours) post infection and protein extracts prepared for ELISA and Western blot analysis. Further, in separate plates, fluorescently tagged CRISPR-AAV payload construct expression vectors are infected into the same cell system and fluorescence intensity is monitored over time. Additionally, cycloheximide blocking and pulse chase experiments are performed in the same cell system.

### B. Destabilizing RNA Sequences

To prepare a destabilized RNA sequence, the CRISPR-AAV payload construct expression vector of Example 2 includes a 3′UTR destabilizing sequence. This destabilized RNA sequence may be used to shorten the half-life of cas9.

One type of 3′UTR destabilizing sequence is AU-rich elements (AREs) and AUUUA motifs. While not wishing to be bound by theory these elements and motifs may be the primary means for mediating mRNA destabilization. Examples of this type of motif are evident in the 3′UTR of human estrogen receptor alpha (hERα) as well as in cytokines, proto-oncogenes and interferon mRNAs.

To determine the effectiveness of the destabilizing RNA sequence, a CRISPR-AAV payload construct expression vector which encodes cas9 and contains a destabilizing sequence, is produced and purified as in example 2. A CRISPR-AAV payload construct expression vector without the destabilizing sequence is used as a control and produced in parallel. The half-life of cas9 mRNA and/or the corresponding protein levels are analyzed over time a predetermined time course in HEK293 cells. Cells are lysed over a time course post infection and RNA and protein extracts are prepared. Half-life of mRNA in both samples is measured by methods known in the art and described herein such as monitoring deadenylation via transcriptional pulsing techniques.

### Example 4. Punctuated Expression of Cas9

In order to study methods to induce transient, burst expression of cas9, a CRISPR-AAV payload construct expression vector of Example 2 which encodes cas9, a DNA binding domain and a transactivating factor is prepared, produced and tested by methods known in the art and described herein. The cas9 sequence is located in the open reading frame of the vector and a DNA binding domain (DBD) and transactivating factor are located in VP2. The transactivating factor may be coupled to the DBD.

The DBD may be a pre-engineered DBD targeted specifically to the promoter used for cas9. While not wishing to be bound by theory, upon expression of VP2 in a biological system, the DBD locates and binds to the cas9 promoter. If the transactivating factor is for cas9 and the transactivating factor is coupled to the DBD, upon DBD binding to the cas9 promoter, the transactivating factor drives expression of cas9.

The nature of the interaction between the promoter and the DBD coupled to a transactivating factor generates a transient, burst expression of cas9. This punctuated expression may be beneficial as it may limit the possibility of side-effects of extended elevated expression of cas9.

To study the burst expression of cas9, a CRISPR-AAV payload construct expression vector with the DBD and the transactivating factor is purified and produced as described in Example 2. As a control, a CRISPR-AAV payload construct expression vector lacking the DBD and transactivating domain is produced in parallel.

Expression of cas9 is measured by methods described herein and known in the art, such as in HEK293 cells for the CRISPR-AAV payload construct expression vector with or without the DBD and transactivating factor. Cells are lysed at different time points and protein extracts are prepared for ELISA and Western blot analysis.

The specificity of cas9 cleavage is confirmed by deep sequencing of samples collected and the extent of dsDNA cleavage by cas9 or cas9-destabilizing domain fusion protein is measured by ligation-mediated purification or genome modification assays such as SURVEYOR. Indel percentage is calculated from the integrated intensities of the undigested PCR product and each of the cleavage products.

1. A composition comprising an AAV particle comprising a polynucleotide encoding at least one payload and at least one CRISPR regulatable element.

2. The composition of claim 1, wherein the serotype of the AAV particle is selected from AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/rh.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, and/or AAVrh.74.

3. The composition of claim 2, wherein at least one payload comprises a dsRNA, siRNA, miRNA or engineered precursor thereof.

4. The composition of claim 2, wherein at least one payload comprises a wild type mRNA.

5. The composition of claim 2, wherein at least one CRISPR regulatable element comprises a cas9 nuclease.

6. The composition of claim 5, wherein the AAV particle further encodes an sgRNA.

7. The composition of claim 6, wherein the sgRNA is located upstream (5′) of the cas9 nuclease.

8. The composition of claim 6, wherein the sgRNA is located downstream (3′) of the cas9 nuclease.

9. A method of synthesizing a CRISPR-AAV particle comprising
a. introducing into competent bacterial cells
i. a payload construct vector comprising a payload and one or more CRISPR regulatable elements flanked on each side by a parvoviral ITR sequence to produce a payload construct expression vector; and
ii. one or more viral construct vector(s) comprising parvoviral rep and/or cap gene sequences under the control of one or more regulatory elements to produce a viral construct expression vector;
b. introducing into viral replication cells
i. the payload construct expression vector produced in step (a.i) to produce a payload construct particle; and
ii. the viral construct expression vector(s) produced in step (a.ii) to produce a viral construct particle; and
c. co-infecting a viral replication cell with the payload construct viral particle produced in step (b.i) and the one or more viral construct viral particle(s) of step (b.ii) to produce a CRISPR-AAV particle.

10. A CRISPR-AAV particle comprising an AAV polynucleotide, said AAV polynucleotide comprising:
(a) at least one payload; and
(b) at least one CRISPR regulatable element.

11. The particle of claim 10, wherein the serotype of the AAV particle is selected from AAV1, AAV2, AAV3, AAV4, AAV5, AAV6, AAV7, AAV8, AAV9, AAV9.47, AAV9(hu14), AAV10, AAV11, AAV12, AAVrh8, AAVrh10, AAV-DJ8 and AAV-DJ.

12. The particle of claim 11, wherein at least one payload comprises a dsRNA, siRNA, miRNA or engineered precursor thereof.

13. The particle of claim 11, wherein at least one payload comprises a wild type mRNA.

14. The particle of claim 11, wherein at least one CRISPR regulatable element comprises a cas9 nuclease.

15. The particle of claim 14, wherein the AAV particle further encodes an sgRNA.

16. The particle of claim 15, wherein the sgRNA is located upstream (5′) of the cas9 nuclease.

17. The particle of claim 15, wherein the sgRNA is located downstream (3′) of the cas9 nuclease.

18. A method of treating a CNS disorder in a subject in need thereof, the method comprising administering to the subject a therapeutically effective amount of a CRISPR-AAV particle comprising an AAV polynucleotide, said AAV polynucleotide comprising:
(a) at least one payload; and
(b) at least one CRISPR regulatable element.

19. The method of claim 18, wherein the CNS disorder is Parkinson's disease.

20. The method of claim 18, wherein the CNS disorder is Friedreich's Ataxia.

21. The method of claim 18, wherein the CNS disorder is Amyotrophic lateral sclerosis (ALS).

22. The method of claim 18, wherein the CNS disorder is Huntington's disease.

23. The method of claim 18, wherein the CNS disorder is spinal muscular atrophy (SMA).

24. A method of regulating the expression of a protein of interest, said method comprising contacting a subject with a CRISPR-AAV particle comprising an AAV polynucleotide comprising:
(a) at least one payload; and
(b) at least one CRISPR regulatable element.

25. The method of claim 24, wherein the at least one CRISPR regulatable element is a DNA binding domain coupled with a transactivation domain.

26. The method of claim 25, wherein the at least one CRISPR regulatable element is located in the VP2 capsid.

27. The method of claim 25, wherein the expression of the protein of interest is increased for at least 2 hours.

28. The method of claim 25, wherein the expression of the protein of interest is increased for at least 6 hours.

29. The method of claim 24, wherein the at least one CRISPR regulatable element is a cas9 endonuclease fused to a destabilizing domain.

30. The method of claim 29, wherein the destabilizing domain is a destabilizing domain from a protein family selected from the group consisting of FK506 Binding Protein (FKBP), E. coli dihyrofolate reductase (DHFR), mouse ornithine decarboxylase (MODC), and estrogen receptors (ER).

31. The method of claim 30, wherein the destabilizing domain is from the estrogen receptor protein family.
